(19) United States
(12) Patent Application Publication
(10) Pub. No.: US 2012/0302852 A1
(43) Pub. Date: Nov. 29, 2012

(54) IMPLANTABLE MEDICAL DEVICE REMOVAL/INSERTION TOOL

(76) Inventor: JEFFREY A. MATOS, NEW ROCHELLE, NY (US)

(21) Appl. No.: 13/455,915

(22) Filed: Apr. 25, 2012

Related U.S. Application Data
(60) Provisional application No. 61/478,563, filed on Apr. 25, 2011.

(51) Int. Cl. A61B 1/32; A61M 25/10; A61B 5/145

(52) U.S. Cl. 600/365; 600/226; 600/203; 604/99.02

(57) ABSTRACT

When replacing an implanted medical device, the physician may have difficulty locating the new unit in exactly the same space or cavity within the body that held the old unit. Various embodiments and combinations of embodiments of a medical removal/insertion tool are proposed which can be inserted temporarily, as “place holders,” in the space of the implanted medical device to be removed, and used to guide the insertion of the replacement unit and to ensure that the replacement unit is properly situated within the body. In its simplest form, the removal/insertion tool has a “first part” which is configured to facilitate the positioning of the new unit in approximately the same space as the old unit, and a “second part” which serves as a handle for gripping, during use, to insert, position and subsequently remove the first part within the body cavity when replacement is complete.

### CROSS-REFERENCE TO RELATED APPLICATION

This present application claims benefit of priority from U.S. Provisional Application Ser. No. 61/478,563, filed Apr. 25, 2011, entitled “IMPLANTABLE MEDICAL DEVICE REMOVAL/INSERTION TOOL”.

### BACKGROUND OF THE INVENTION

When an implantable pacemaker battery is substantially depleted of energy, the entire pacemaker gets replaced (not just the battery—because the battery is hermetically sealed inside the pacer)—an event that occurs about every 7 years.

A pacemaker is shaped like an elliptical silver dollar, but has about 2.5 times the thickness of an actual silver dollar.

Occasionally there is a problem with the positioning of a new pacemaker unit wherein the new unit is not put exactly where the old one was, but in a location which:
is less deep (from the skin surface) than the old one, but
has similar coordinates to the old one along the right-left axis, and
has similar coordinates to the old unit along the head-foot axis.

When such a problem occurs, it may cause patient discomfort, and may be a cosmetic problem—because the new (i.e. replacement) unit is not buried as far below the surface of the skin, and at times it sticks out noticeably. In extreme cases, a unit that is too near to the skin surface will, over a period of time, ultimate break the surface of the skin and need to be entirely replaced.

The problem (shown in the sequence of FIGS. 1A to 1B) may occur because once the surgeon pulls out the old (i.e. depleted) pacemaker unit, the tissue on one side of the space initially occupied by the old unit (FIG. 1A) approaches and may contact the tissue that is on the other side of that space, resulting in the space substantially closing itself up (FIG. 1B). As the surgeon tries to insert the new unit, he or she may get it back into the old space, or may get into a newly created space which is nearer to the surface of the skin, or may insert the unit so that it is partially situated in the old space and partially situated in a new space.

Everything stated herein about pacemakers is applicable to implantable defibrillators, implantable cardioverter defibrillators, implantable insulin pumps, implantable glucose sensing devices, implantable nerve and brain stimulators, implantable drug infusion pumps and other implantable medical devices.

### SUMMARY OF THE INVENTION

The need exists, therefore to provide the surgeon who implants and replaces such devices with means to assure that the new device is situated in an appropriate location and orientation within a patient. In the case of device location, often the preferred location is where the old device was situated.

The current invention provides a variety of means to accomplish this task. They include

(A) A Spatula-shaped device, and variants, for marking the place of a previously implanted device prior to its removal;
(B) A glove-like device with tearable section for guidewire introduction. This device may have inboard sheaths to allow for the placement of guidewires.
(C) A glove-like device with outboard sheaths for guidewire introduction;
(D) A 3 component system with inner, intermediate, and large bore outer sheath. The intermediate and outer sheaths are shaped like the implantable device (“IMD”).
(E) A first type of balloon device—inserted over guide wire, which fills the old pocket. The balloon may deflate as the IMD is advanced into pocket.
(F) A second type of balloon device—also inserted over guide wire. The IMD is inserted into the balloon. The inflated balloon allows for easing the IMD into the cavity. The balloon is removed by its tear-away feature.
(G) The IMD has one or more holes that accommodate guiding devices. The holes do not communicate with the internal electronics of the IMD.
(H) An elastic-like belt is strapped onto the IMD. The belt has receptacles for at least one guiding device. After implant positioning, the belt and guide(s) are removed.
(I) A long flexible guidewire is inserted into the cavity. It curls around in the cavity and eventually the distal end emerges. This leaves both the proximal and the distal ends sticking out of the cavity. A large-bore intermediate guiding device with 2 parallel holes is slid in over these two protruding guide wire ends. A large-bore sheath surrounds the intermediate guiding device. The IMD is then inserted via the large-bore sheath.

Combinations of these approaches may be best suited for any individual patient. For example, at the time of device replacement, the glove device described hereinabove in paragraph (C) may be ideal for initially securing access to a previously formed pocket. Following the placement of the distal end of a guidewire into the pocket by the glove device, the apparatus and method described in paragraph (I) hereinabove may be used to place a large sheath into the pocket, which, in turn, facilitates the placement of the IMD.

Another example of combined approaches is guidewire placement by a glove of the type described in paragraph (B) hereinabove, followed by the insertion of either the balloon device of paragraphs (E) or (F) hereinabove.

Numerous other combinations of approaches will be evident to those skilled in the art.

### BRIEF DESCRIPTION OF THE DRAWINGS

FIG. 1, comprising FIGS. 1A and 1B, shows the prior art.

FIG. 2A is a top view of one type of insertion/removal device.

FIG. 2B is a side view of one type of insertion/removal device.

FIG. 2C is a schematic view of the device of FIGS. 2A and 2B in place in an IMD pocket in a patient undergoing an IMD insertion or replacement procedure.

FIG. 3A is a cross sectional/schematic view of the steps involved in using a 1-element version of the device of FIGS. 2A and 2B.

FIG. 3B is a cross sectional/schematic view of the steps involved in using a 2-element version of the device of FIGS. 2A and 2B.

FIG. 3C is a cross sectional/schematic view of the steps involved in using a 3-element version of the device of FIGS. 2A and 2B.

FIG. 3D is a cross sectional/schematic view of the steps involved in using a 4-element version of the device of FIGS. 2A and 2B.

FIG. 3E is a cross sectional/schematic view of the steps involved in using another type of 4-element version of the device of FIGS. 2A and 2B.

FIG. 4A is a side view of the 2-element device of FIGS. 2A and 2B, showing a removable device element, and an attachment device.

FIG. 4B shows a detail of the removable element of the device of FIG. 4A.

FIG. 4C shows a detail of another removable element of the device of FIG. 4A.

FIG. 5 is a side view of the 4-element described in FIG. 3D, showing two removable device elements, and an attachment device.

FIGS. 6A and 6B show closed and open schematic view of a 2 element device which may be expanded laterally after insertion into a pocket.

FIGS. 7A and 7B show closed and open schematic view of another 2 element device which may be expanded laterally after insertion into a pocket.

FIG. 8A shows a schematic view of a 3 element device which may be expanded laterally after insertion into a pocket.

FIG. 8B shows a schematic view of a 5 element device which may be expanded laterally after insertion into a pocket.

FIGS. 9A and 9B show schematic views of two types of the 1-element device of FIGS. 2A and 2B, with guiding devices.

FIG. 10A is a frontal view of an IMD with guiding receptacles placed laterally.

FIG. 10B is a perspective view of the IMD of FIG. 10A, during insertion over guiding devices, into an IMD pocket.

FIG. 10C is a top/oblique view of an IMD showing both a guiding receptacle not contiguous with the IMD lateral border, and another guiding receptacle which is contiguous with the lateral border.

FIG. 10D is a side/schematic view of the device of FIG. 10C.

FIG. 11A is a side view of an IMD with an attached implant/removal-facilitating belt, oriented horizontally, with two lateral guiding receptacles on the belt.

FIG. 11B is a perspective view of the IMD of FIG. 11A, during insertion over guiding devices, into an IMD pocket.

FIG. 11C is a side/perspective view of an IMD with an attached implant/removal-facilitating belt, oriented vertically, with one lateral guiding receptacle on the belt.

FIG. 12A is a side view of an IMD with lateral indentations to more securely accommodate the horizontal belt shown in FIG. 11A.

FIG. 12B is a side view of an IMD with lateral clips to facilitate implant and/or removal.

FIG. 12C is a schematic bottom and side view of an IMD with both implant/removal-facilitating protuberances and depressions.

FIG. 13 is a schematic view of the insertion and operation of one type of balloon device.

FIG. 14 is a schematic view of the insertion and operation of another type of balloon device.

FIGS. 15A and 15B show a side and top view of an exemplary IMD.

FIG. 15C shows a cross-sectional view of (i) a 1-element inner guiding device, (ii) an intermediate apparatus, and (iii) an outer sheath, for facilitation of the implantation of the IMD of FIGS. 15A and 15B.

FIG. 15D is a cross sectional view showing the IMD of FIG. 15B. inside of the outer sheath of FIG. 15C.

FIG. 15E is a perspective view of the apparatus of FIG. 15C situated such that the distal ends of each of the three components is within a space in the body for IMD placement.

FIG. 16A shows another top view of exemplary IMD.

FIG. 16B shows a cross-sectional view of (i) a 3-element inner guiding device, (ii) an intermediate apparatus which accommodates the three inner guides, and (iii) an outer sheath, for facilitation of the implantation of the IMD of FIG. 16A.

FIG. 16D is a perspective view of the apparatus of FIG. 16B situated such that the distal ends of each of the five components is within a space in the body for IMD placement.

FIG. 17 is a perspective view of a guidewire which has been caused to form a semi-circular loop in an IMD pocket, also showing an intermediate apparatus which accommodates both ends of the guidewire, and an outer sheath.

FIG. 18 shows a perspective view of a glove device for introducing guiding devices into an IMD pocket.

FIG. 19A is shows a side/schematic view of one finger segment of a glove-device with an outboard guiding receptacle for a guidewire.

FIG. 19B is a schematic view of an outboard guiding receptacle placed at the top of the glove-device, showing a guidewire within the receptacle.

FIGS. 19C and 19D are schematic views of an outboard guiding receptacle placed at the bottom and at the side of the glove-device, each figure not showing a guidewire within the receptacle.

FIG. 19E is a schematic view of two outboard guiding receptacles in top/lateral positions on the glove-device, showing guidewires within the receptacle.

FIG. 19F is a schematic view of an outboard guiding receptacle placed between two finger segments of the glove-device, showing a guidewire within the receptacle.

FIG. 20 is a side/schematic view of a glove-device without guiding receptacle, with tearable segment, for placement over a gloved hand of a surgeon.

FIG. 21A is a side/schematic view of a glove-device with inboard guiding receptacle, with tearable segment.

FIG. 21B is a schematic view of an inboard guiding receptacle placed at the top of the glove-device showing a guidewire within the receptacle.

FIGS. 21C and 21D are schematic views of an inboard guiding receptacle placed at the bottom and at the side of the glove-device, each figure not showing a guidewire within the receptacle.

FIG. 21E is a schematic view of two outboard guiding receptacles in top/lateral positions on the glove-device, not showing guidewires within the receptacle.

FIG. 22 is a top/schematic view of a glove-device without guiding receptacle, with tearable segments on each of two finger portions, for placement over a gloved hand of a surgeon.

FIG. 23A shows a linear tearable segment of the device of FIG. 22

FIG. 23B shows a curved tearable segment of the device of FIG. 22

FIG. 23C shows another curved tearable segment of the device of FIG. 22

FIG. 24 is a schematic view of a glove-device with multiple guiding receptacles within the glove material.

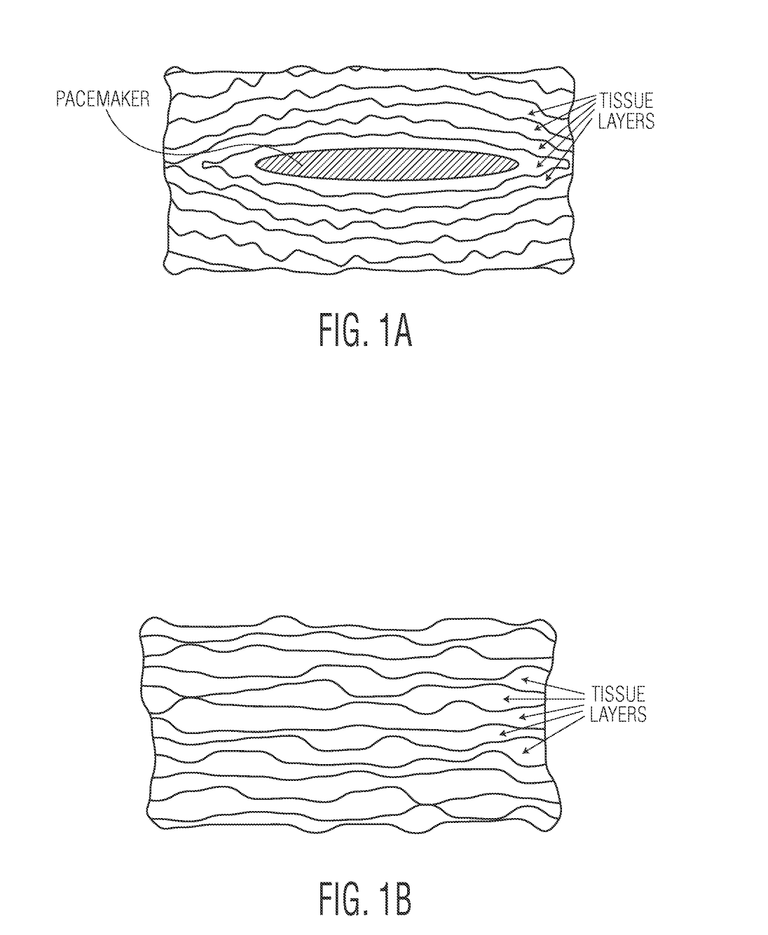

### DETAILED DESCRIPTION OF THE PREFERRED EMBODIMENTS

The Removal/Insertion Tool (hereinbelow referred to as the “R/I tool”) facilitates the replacement of the unit with the depleted battery (hereinbelow referred to as the “old unit”) with a unit powered by an adequate battery (hereinbelow referred to as the “new unit”). Specifically, the R/I tool facilitates the positioning of the new unit in approximately the same space as the old unit.

In the text and figures, pacemaker is at times referred to as “pacer.” The words and abbreviations “pacer”, “pacemaker” “implantable medical device” and “IMD” are each intended to include all manner of implantable medical device as delineated in the previous paragraph. Also in the figures, the R/I tool is in some instances referred to as “Insertion/Removal tool” and is also referred to as “removal tool”. Broken lines indicate the edge of an element which lies behind that of another element as shown in the figure.

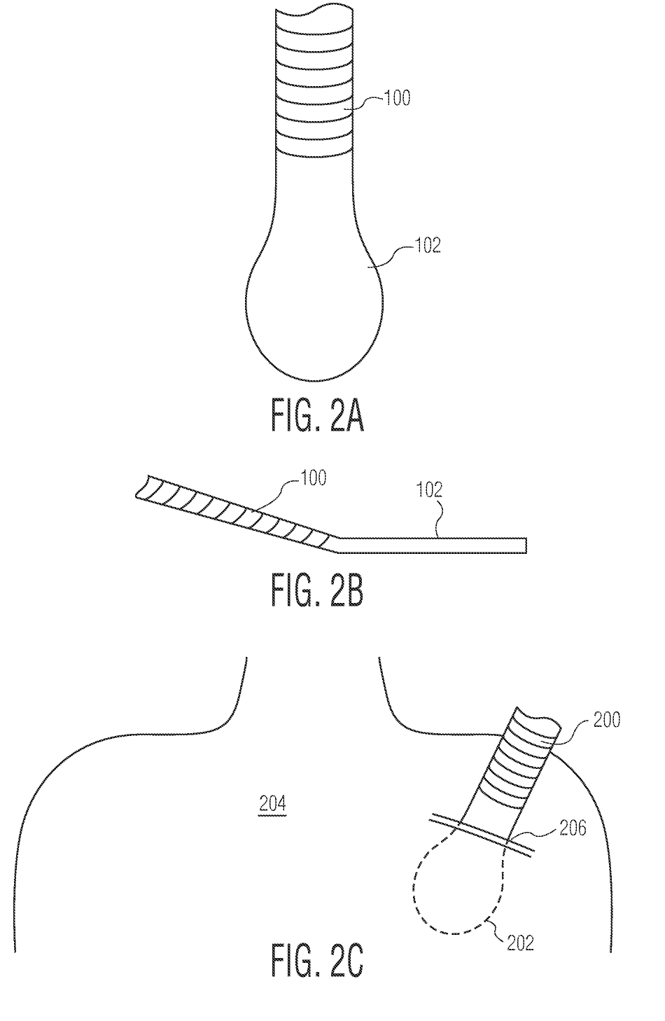

The invention, the medical device R/I tool is shown in FIGS. 2A and 2B, with
(a) a handle 100, attached to
(b) a roughly flat segment 102, which may have a shape similar to that of the pacemaker, but is considerably thinner.

The flat segment gets inserted into the tissue space before the old pacemaker is pulled out. A handle which is curved, or one which is angled with respect to the flat segment facilitates the insertion of the R/I tool.

FIG. 2C shows a partial view of a patient 204 lying supine (i.e. on a procedure table which is not shown in the figure) with the R/I tool inserted into left pectoral incision 206. The handle 200 is visible above the surface of the patient's skin, while the flat portion 202 is shown with broken lines indicating its location beneath the surface of the skin.

The R/I tool may also be used for a de-novo device implantation, to facilitate the entry and positioning of a medical device which is being implanted, once a pocket has been created for it, beneath the skin surface.

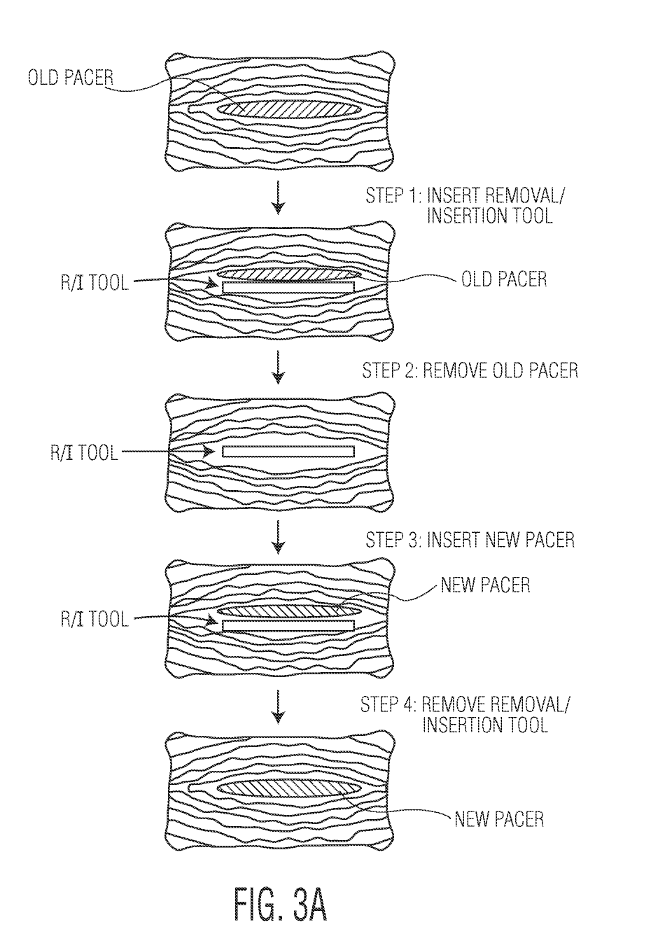

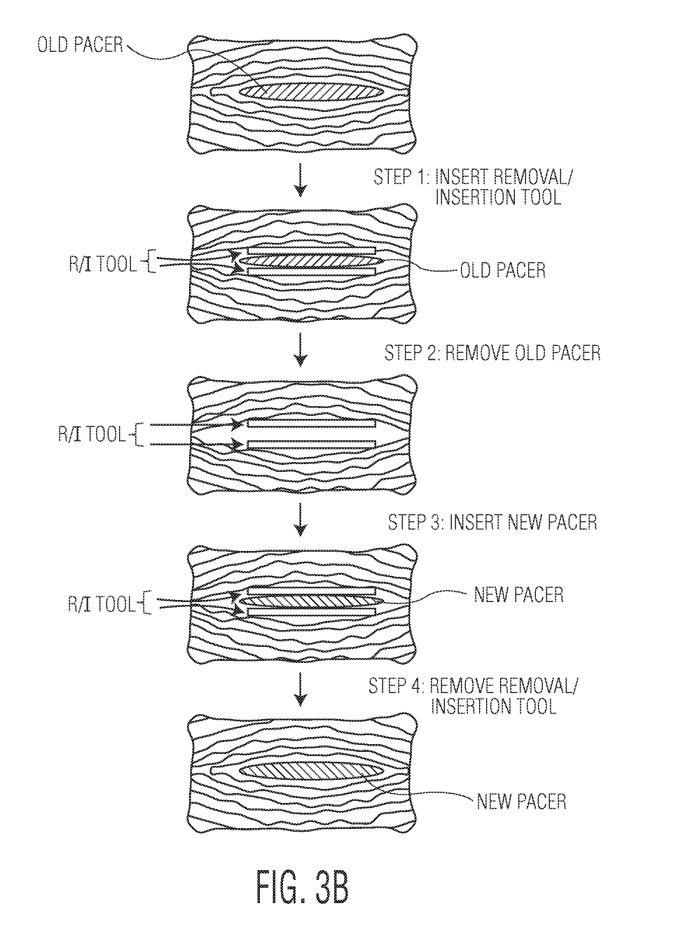

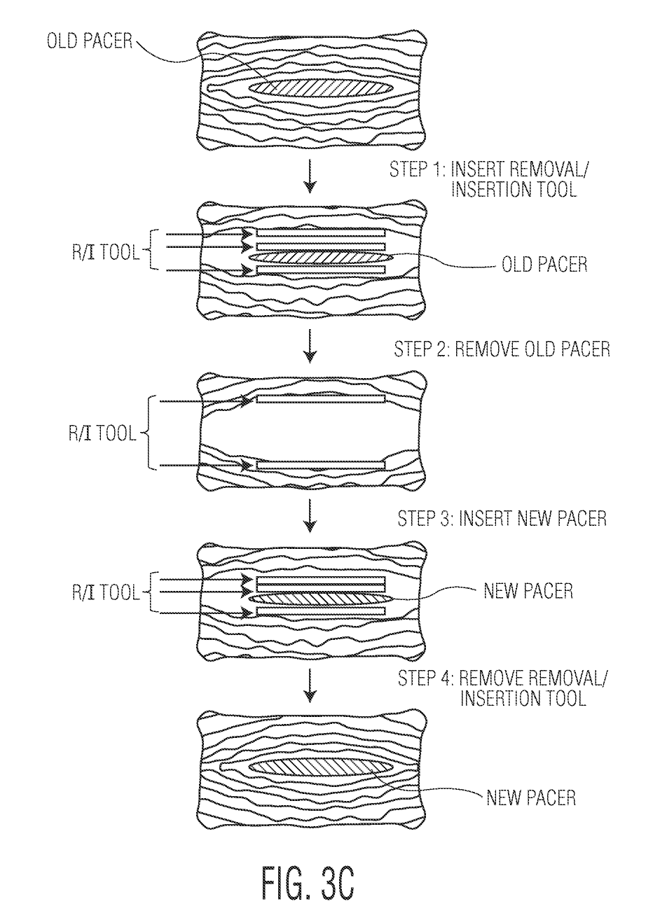

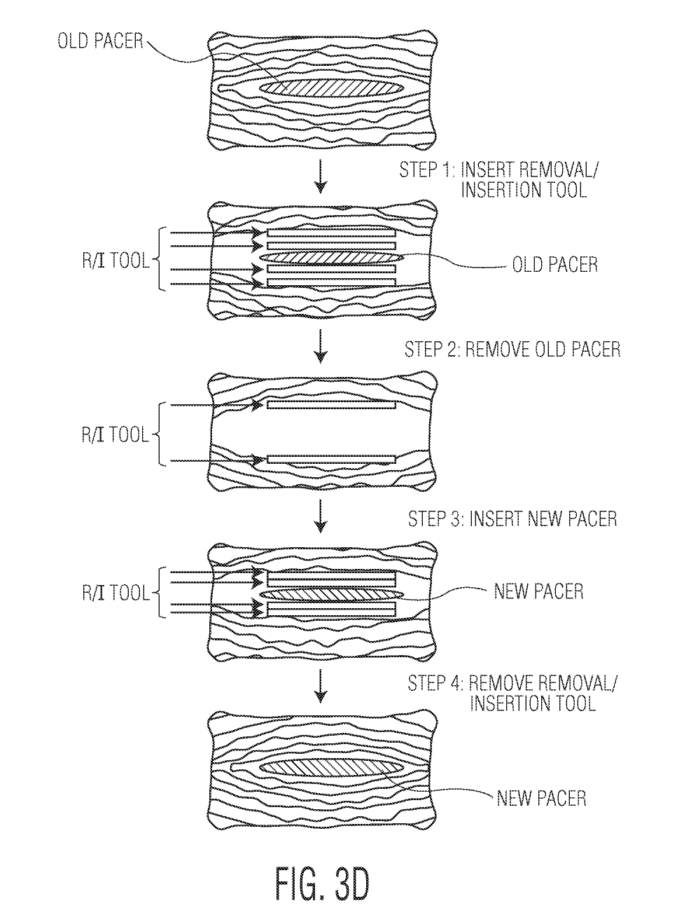

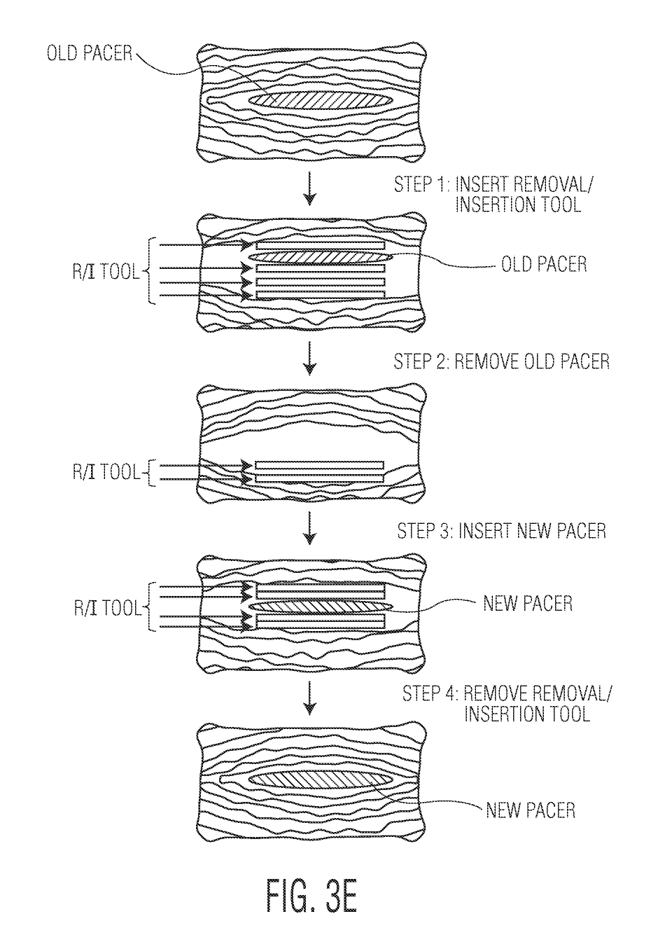

FIG. 3A and Table 1 indicate the sequence of events involved in the use of the R/I tool.

TABLE 1

| Step # | Action |
|---|---|
| 1 | Insert R/I tool, below (i.e. deep to) the old pacemaker |
| 2 | Remove old pacemaker |
| 3 | Insert new pacemaker above (i.e. nearer to the skin |
| | surface than) the R/I tool |
| 4 | Remove the R/I tool |

Referring to Table 1 and FIG. 3A, after the R/I tool is inserted (Step #1), the old pacemaker is removed (Step #2), and the new pacemaker is slid in along/over the R/I tool (Step #3)—allowing the new unit to enter the same space that the old unit occupied. Finally (Step #4), the R/I tool is removed after the new unit has been successfully inserted.

The process of pacemaker replacement may also be accomplished using an R/I tool with two elements (or by using two such R/I tools), as shown in the sequence in FIG. 3B. One element of the R/I tool is inserted below the pacemaker (i.e. deep to the pacemaker, with respect to the skin surface), and one element of the R/I tool is inserted above it (i.e. nearer to the skin surface than the pacemaker). The below-the-pacemaker R/I tool element and the above-the-pacemaker R/I tool element are joined at a handle.

FIG. 3C schematically illustrates the performance of a 3 element version. In the variation shown in the figure, all 3 elements are introduced into the pocket, two above and one below the pacer (or, as indicated hereinabove, and as is applicable hereinabove and hereinbelow, implantable medical device). The pacer is then removed along with the middle element (which may be able to grip the device by a hook type apparatus [not shown in this figure] either on the pacer or on the tool). The remaining two elements “keep the place” of the pacer. In Step 3, a new pacer is inserted between the two remaining elements. And in Step 4, the remaining elements of the tool are removed.

FIG. 3D schematically illustrates the performance of one type of 4 element version. In the variation shown in the figure, all 4 elements are introduced into the pocket, two above and two below the pacer. The pacer is then removed along with the middle two elements. The remaining two elements “keep the place” of the pacer. In Step 3, a new pacer is inserted between the two remaining elements. And in Step 4, the remaining elements of the tool are removed.

The 4 element apparatus functions similarly in FIG. 3E, except that in this variation one element is introduced above and three below the pacer. The pacer is then removed along with the upper two elements. The remaining two elements “keep the place” of the pacer.

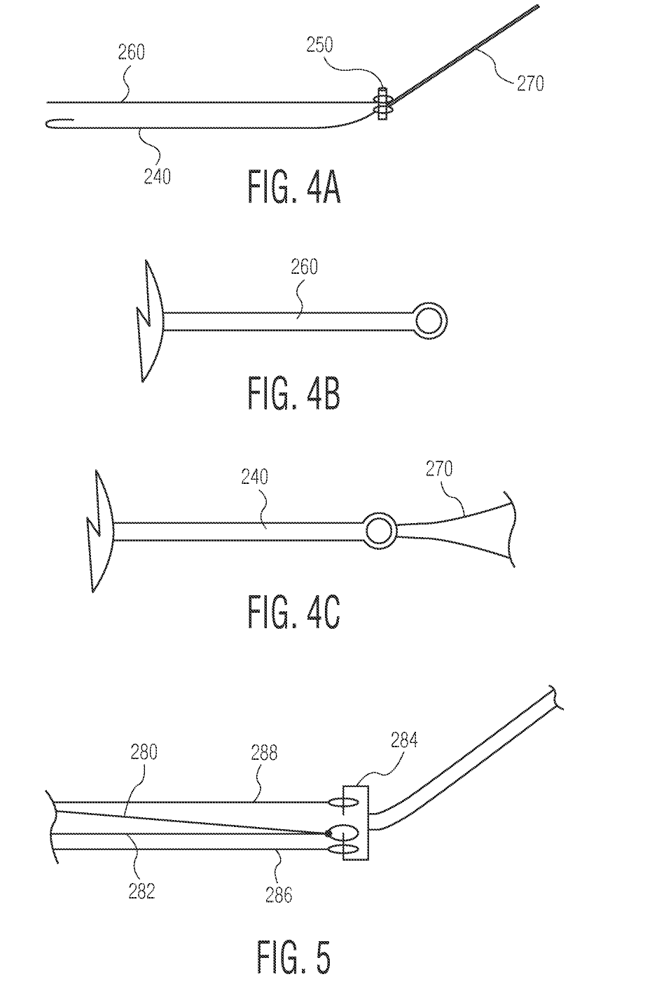

FIG. 4A shows the 2-element device of FIGS. 2A and 2B, with a removable device element 240, and an attachment device 250. 240 has a hook at its end, for mating with an appropriately shaped depression in an IMD. 250 may be any one of a variety of fastening devices allowing detachment known in the art. It may have a screw-type element, or not have threading. FIG. 4B shows a detail of FIG. 4A, illustrating a top view of removable element 260. FIG. 4C shows a top view of another removable element 240, attached to handle 270.

FIG. 5 is a side view of the 4-element described in FIG. 3D, showing two removable device elements 280 and 282, and an attachment device 284. In this variation, the pacer (IMD) would be removed along with the 280/282 composite element gripping the pacer. Elements 288 and 286 remain behind, as a placeholder. Attachment device 284 allows adjustment of the distance between the upper and middle elements and between the middle and lower element.

The flat portion of the R/I tool may be less wide than the pacemaker (facilitating insertion into the patient) or the shape and/or width of the R/I tool may be designed to conform to the shape of the old unit or the new unit. Each manufacturer might therefore have their own R/I tools, since there is no universal pacemaker (or other implantable device) shape. In fact, a device manufacturer may have multiple device shapes. Manufacturers' kits would likely contain R/I tools of different thicknesses (as well as different shapes).

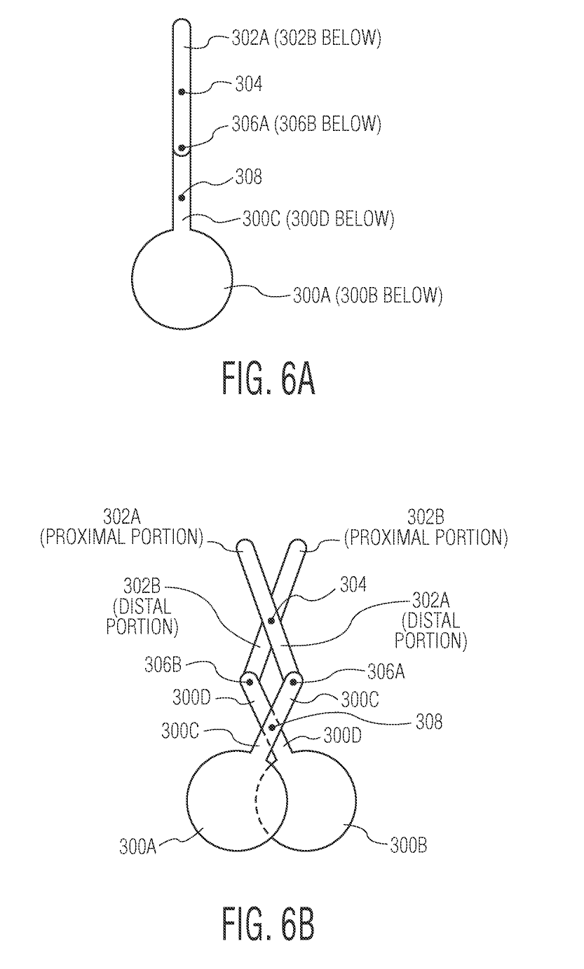

FIGS. 6A and 6B show an embodiment of the invention in which the shape of the R/I tool is able to be manipulated once it is inside the patient, in order to more fully conform to the shape of the pacemaker, after the tool has been inserted into the patient.

FIG. 6B shows the extended configuration of an R/I tool with two flat portions 300A and 300B, which are moveable with respect to each other; FIG. 5A shows the collapsed configuration. The device is inserted into the patient in the collapsed configuration, in which element 300B lies below 300A. (Elements 300A and 300B serve an analogous function to 100A of FIG. 2A.) Once inside the patient, the proximal portions handles 302A and 302B are moved apart, which causes the device to assume the extended configuration shown by FIG. 6B. As the proximal portions of 302A and 302B are moved, the central portion of these members is constrained by pivot apparatus 304, resulting in the distal portions of 302A and 302B also moving apart. The distal portion of 302A is linked to member 300C via pivot apparatus 306A; 300C has a fixed relationship to flat portion 300A; Similarly, the distal portion of 302B is linked to member 300D via pivot apparatus 306B; 300D has a fixed relationship to flat portion 300B. The central portion of members 300C and 300D is constrained by pivot apparatus 308. Thus, by a dual scissoring action about each of pivot apparatus 304 and 308, the movement of the proximal portion of 302A away from 302B results in the separation of 300A from 300B, thereby allowing the composite of 300A and 300B to transition from a circular shape (in the collapsed configuration) to a somewhat elliptical shape (in the extended configuration). (As referred to herein, the term “elliptical” is not intended to have the precise geometric definition of an actual ellipse, but rather to indicate a somewhat oval shape which is roughly similar to that of some pacemakers and implantable defibrillators.)

Many other configurations of a variable shape tool will be apparent to those skilled in the art. For example:

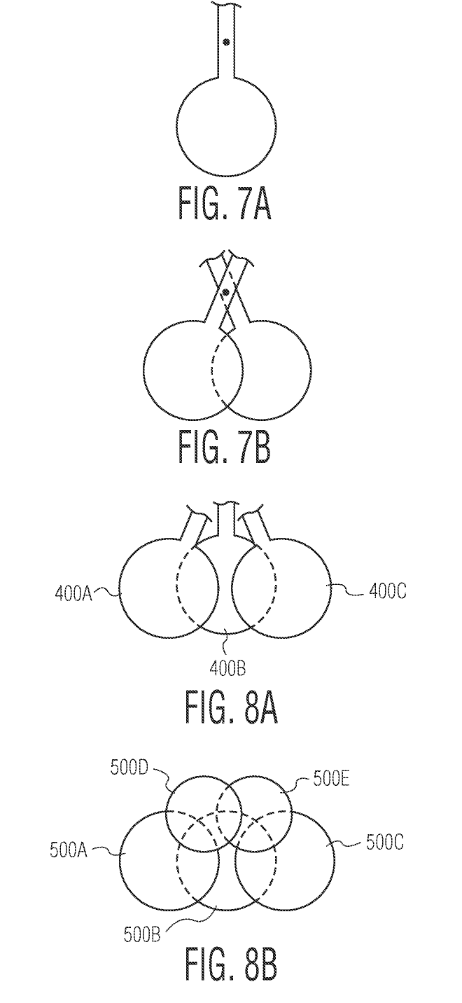

Devices in which a larger number or a smaller number of pivot points are possible. FIGS. 7A and 7B show a version with only a single pivot point.

Devices in which elements 300A and 300B are not circular are possible. In general, the shape of such elements will be such that the composite shape may approximate that of the old device, the new device or both.

The position of the pivot points along each of elements 502A and 502B, and along each of elements 500C and 500D as shown in the figure are exemplary, and the positioning of these pivot points at relatively more proximal or more distal locations are possible.

Embodiments with 3 or more flat portions are possible, including, for example, that shown in FIG. 8A (3 elements [400A, 400B and 400C] in a row) and that shown in FIG. 8B (5 elements in 2 rows [500A, 500B, 500C, 500D and 500E]). Many other such variations are also possible.

Embodiments in which a releasable locking apparatus secures the R/I tool in either or both of the extended of the collapsed configurations. Many forms of locking apparatus will be apparent to those skilled in the art.

Additional features of the R/I tool are:
Variations in the handle geometry (see FIGS. 2A and 2B) facilitate manipulation of the flat portion of the R/I device. Handles of various shapes, and with variable (or no) angulation with respect to the flat portion are possible. A device with a removable handle is possible.
Materials have been designed for medical procedures which are especially slippery. These would be advantageous for coating the side of the inserted/flat portion of the R/I tool which is contacts the pacemaker. Alternatively such a slippery coating may be applied to both sides of the flat portion.

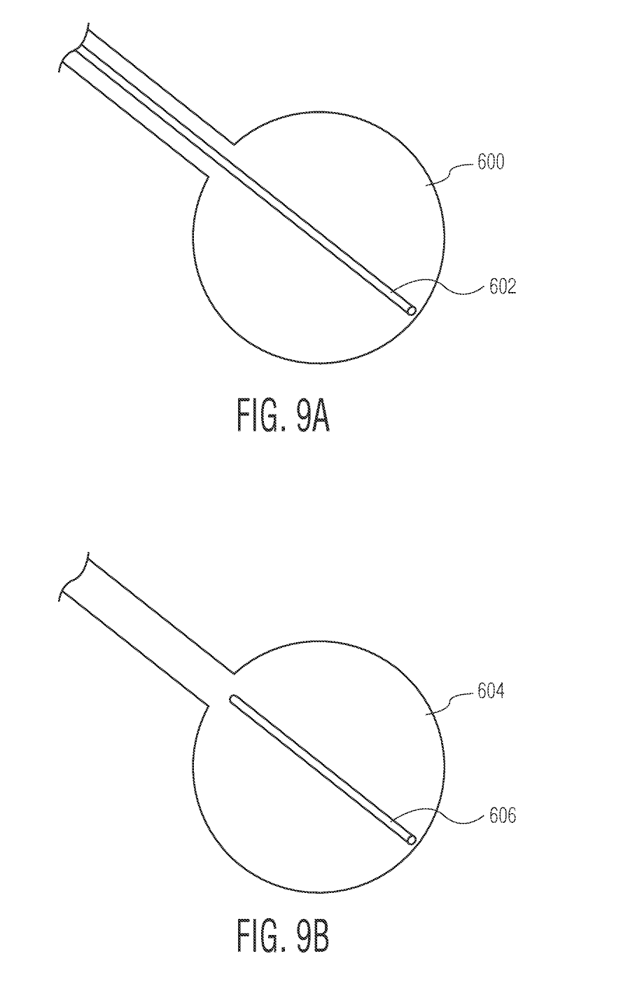

FIG. 9A shows a variation in which tool 600 has an attached receptacle 602 for a guiding device. The guiding device allows introduction of a guide wire into the IMD pocket, which thereafter allows for the introduction of other hardware into the pocket, as discussed hereinbelow. FIG. 9A shows a variation in which the receptacle extends onto the handle. FIG. 9B shows a variation in which a shorter receptacle 606 is only on the proximal portion of the tool 604.

Another approach to facilitating device placement is to secure access to a pocket with appropriately placed guiding devices, and then slide an IMD (with receptacles for the guiding devices) into the pocket using the guiding devices.

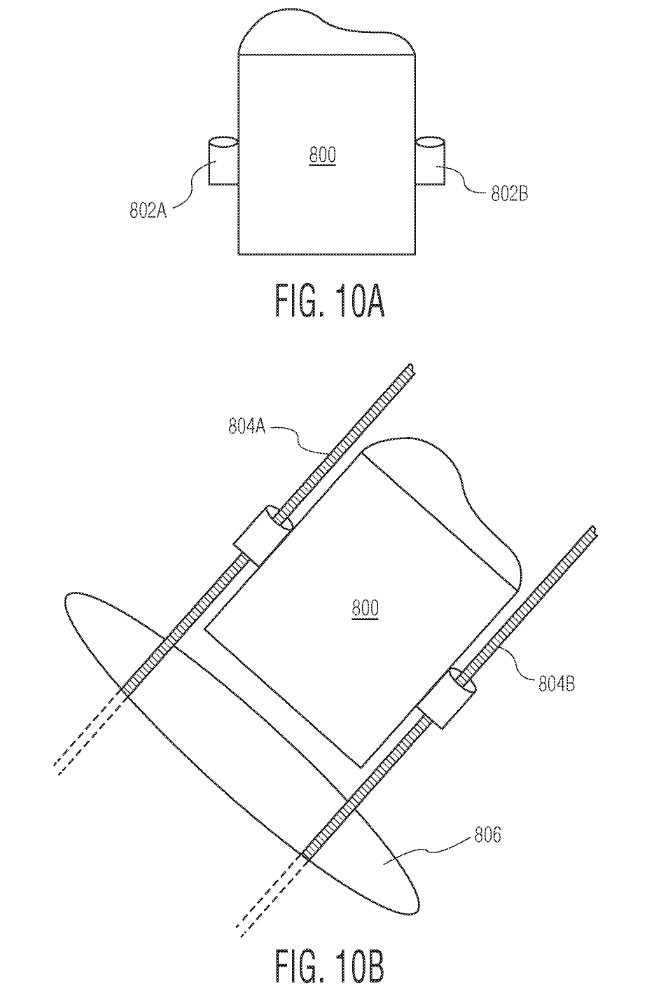

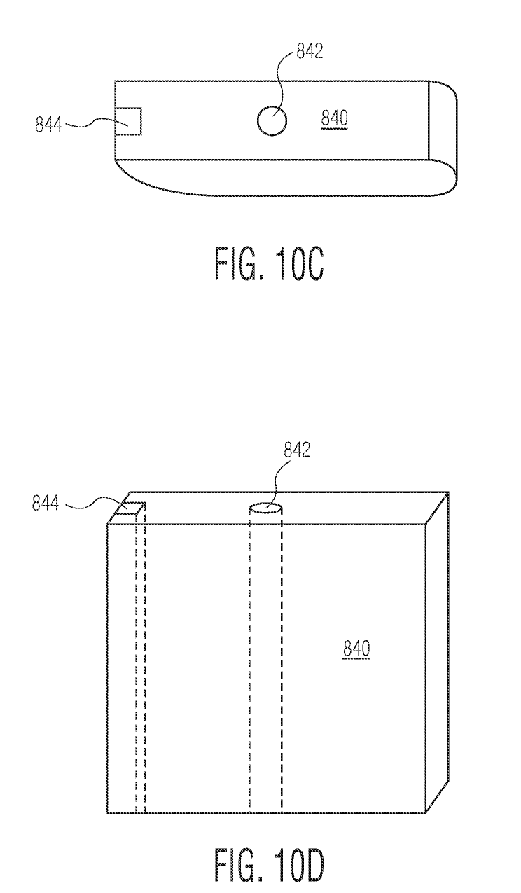

One variation of this approach is shown in FIG. 10. In FIG. 10A, IMD 800 has two guiding receptacles 802A and 802B which are part of the device housing. Although two are shown in the figure, any number and placement of the receptacles is possible. FIG. 10B shows the IMD of FIG. 10A being slid into IMD pocket 806 over guiding devices 804A and 804B, 804A and 804B will be sufficiently stiff to execute their support function as the IMD is advanced into the pocket.

FIGS. 10C and 10D show geometric variants of the aforementioned device. 842 represents a centrally placed round hole in IMD 840, and 844 represents a laterally placed rectangular hole, neither of which is not in physical communication with the device's inner contents. Holes of other shapes and placements are possible, as are holes which run in non-vertical directions.

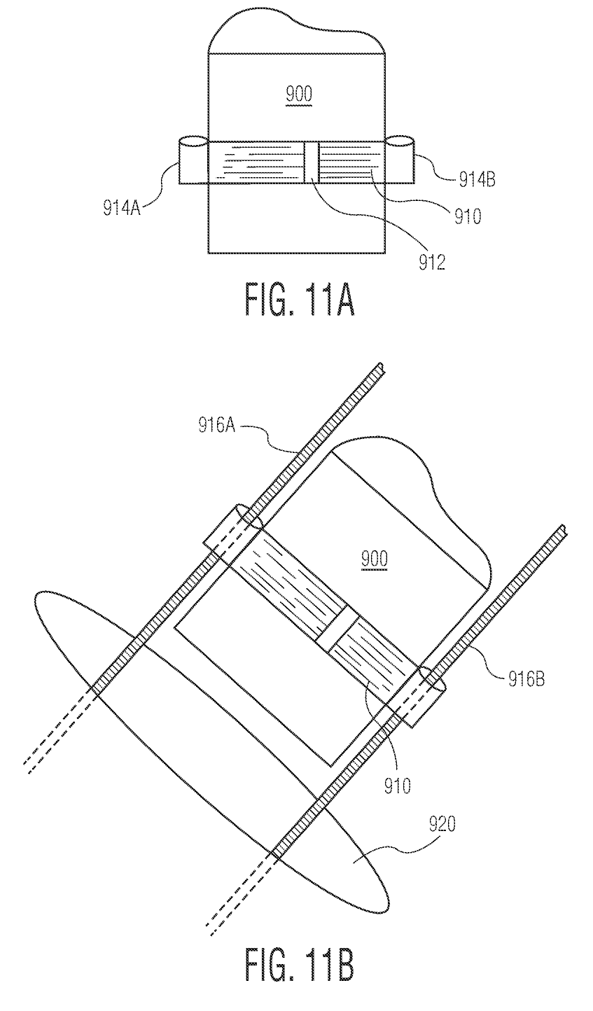

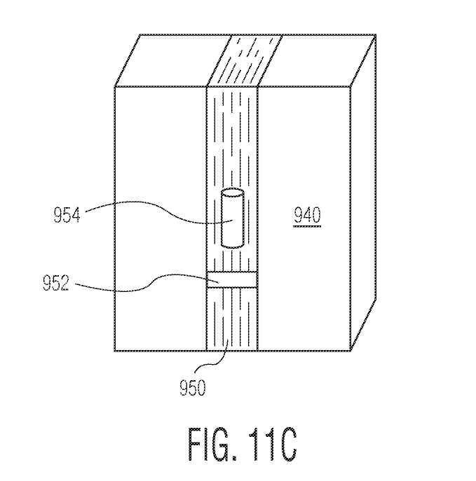

FIGS. 11A-C show an arrangement in which the guiding receptacles are not on the IMD proper, but are attached to a removable belt. In FIGS. 11A and B, IMD 900 is fitted with belt 910 made of stretchable material that tightly grips the device. Fastener 912 allows for any of size and/or tension adjustment. Two guiding receptacles 914A and 914B are shown, which accommodate guiding devices 916A and 916B respectively, for guiding the IMD into pocket 920. After deployment of the IMD, the belt is removed. FIG. 11C shows a vertically oriented belt 950, with fastening device 952 and guiding receptacle 954 surrounding IMD 940. The deployment of this version is analogous to that of the horizontally oriented belt version.

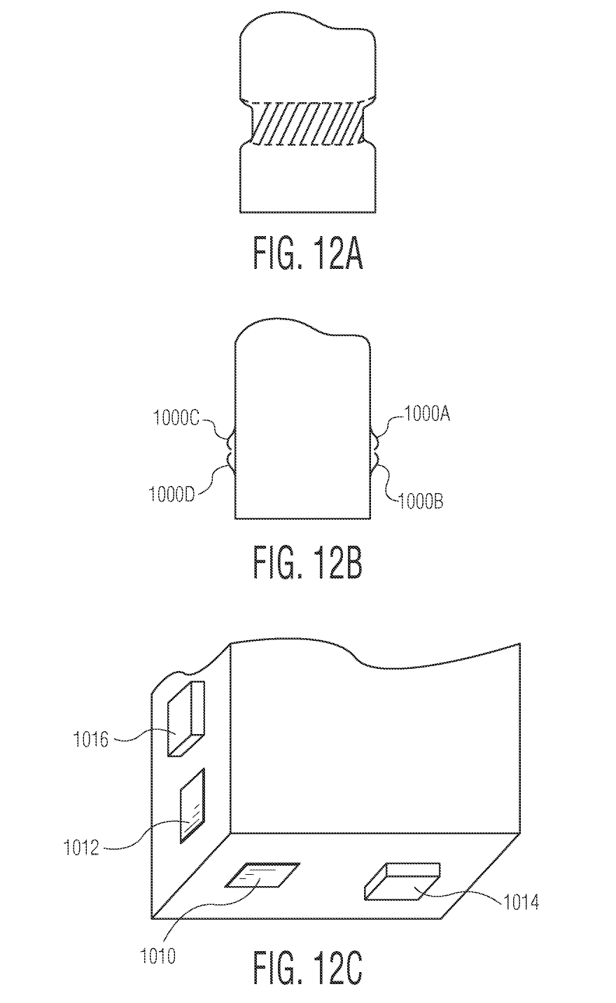

FIG. 12A shows an IMD housing design which further secures the horizontal belt, using depressions in the lateral portion of the housing. An analogous version of the housing, not shown, would function with a vertically oriented belt.

FIG. 12B shows clips 1000A-D which are part of the IMD housing. Each pair may be a single entity, or the four may be separate. A wide variety of geometric shapes, positions, locking mechanisms, placements and clip number are possible.

FIG. 12C shows additional geometric IMD variations to facilitate insertion and removal. 1010 and 1012 are depressions in the IMD, 1014 and 1016 are elevations. A wide variety of shapes, heights, depths, placements and number are possible.

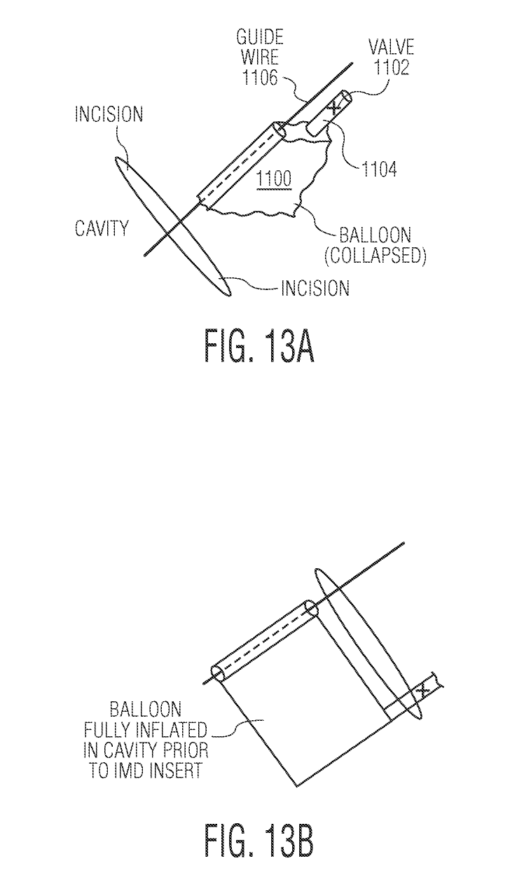

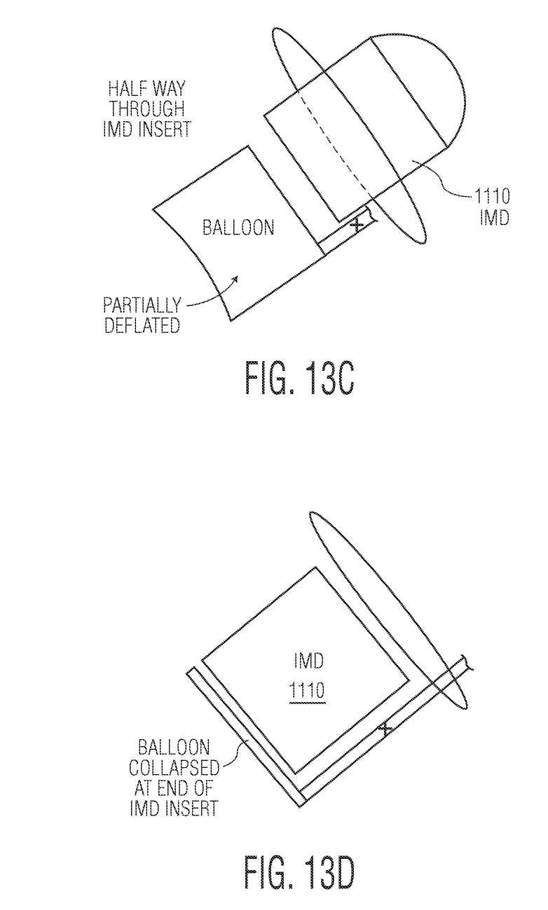

Balloon devices have the advantage of being able to inflate to the shape of an already existing cavity. FIG. 13 shows a scenario in which a balloon device 1100 is first inserted into a pocket to mark/maintain its location. A guide wire 1106 aids in the placement of the balloon device. The balloon device has a valve 1102 which keeps inserted liquid or gas inside and allows its measured removal. The balloon device has a neck 1104 which facilitates balloon device removal after the task is done. After balloon insertion in the deflated state (Panel I) the balloon is inflated (II). Next the IMD 1110 is inserted (III). One approach is to gradually collapse the balloon as the IMD is inserted. In (IV), the balloon is fully collapsed, and is removed. In one embodiment, the balloon may be inserted in the collapsed state before the IMD is removed, and inflated as it is removed.

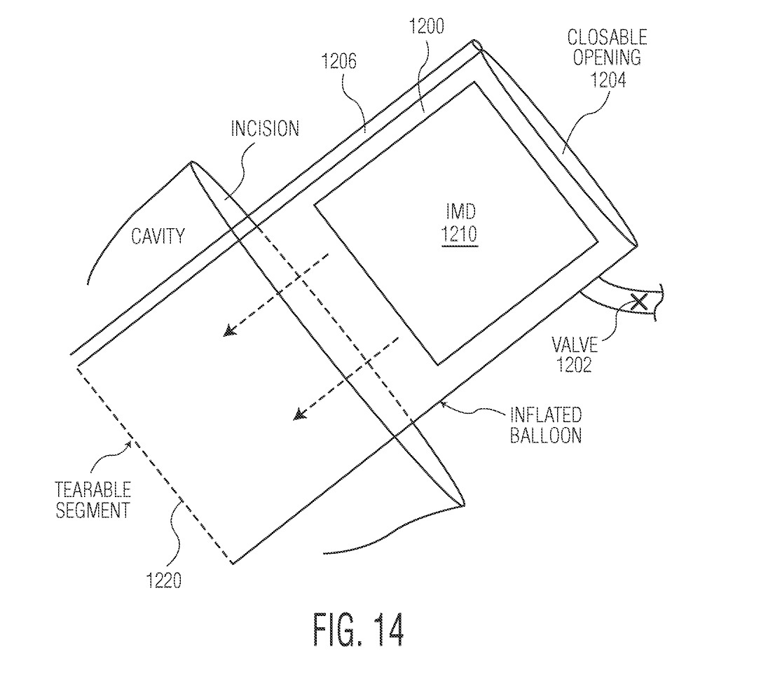

FIG. 14 shows an alternative type of balloon device 1200. In this arrangement, the IMD 1210 is inserted through a closable opening 1204 in the balloon. The opening is closed. The balloon is inflated. The balloon with its IMD contents is then manipulated into the IMD cavity/pocket, and the IMD is moved to the most distal region of the balloon interior. Valve 1202 controls fluid Ingress/egress. The balloon plus IMD may be fully inflated or partially inflated at the time of insertion. Optional guidewire receptacle 1206 may also facilitate insertion. Following IMD placement tearable segment 1220 allows removal of the balloon.

The balloons of FIGS. 13 and 14 may either be generic, or may be specifically tailored to the shape (or a modification thereof) of the IMD.

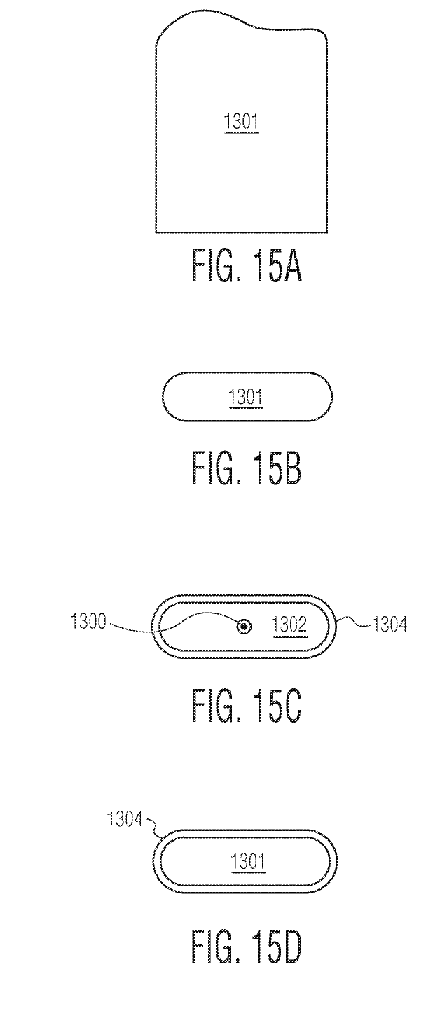

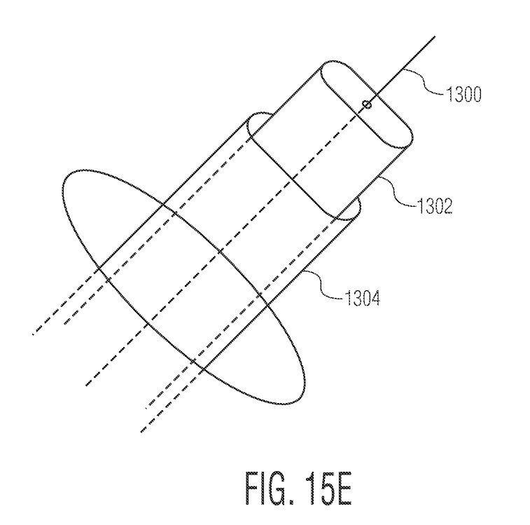

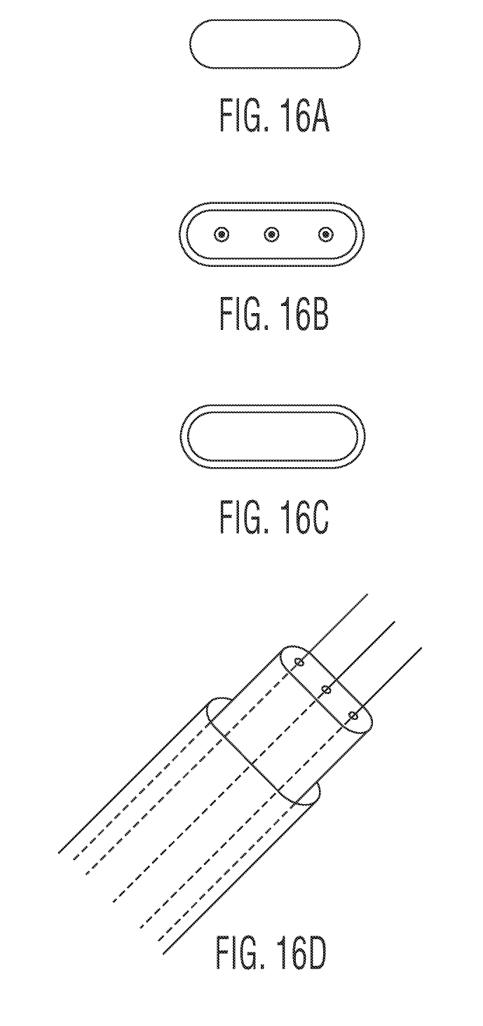

FIGS. 15A-E show the insertion of an IMD using a technique known in the art of vascular medicine, in which an initial thin guidewire is placed, following which a combination of an intermediate diameter cylindrical device (for mechanical support) and outer sheath are advanced into the vessel over the guidewire. The guidewire and intermediate device are then removed, leaving the sheath in place to allow for the insertion of devices far larger than the initial guidewire. In the invention of FIG. 15, the technique and hardware are modified to conform to the shape of an IMD in a projection in which it is to be inserted. FIGS. 15A and 15B show the IMD 1301. FIG. 15C shows a cross sectional view of the guidewire 1300 plus intermediate apparatus (“IA”) 1302 and outer sheath 1304 situated one within the other. These three elements are shown placed into the IMD pocket in FIG. 15E: an initial step of guidewire 1300 insertion would be followed by a second step of IA plus outer sheath insertion, followed by a third step of guidewire and IA removal. The IMD 1301 is then inserted into sheath 1304, as is shown in the cross sectional view of FIG. 15D; and the IMD is advanced into the most distal portion of the sheath/pocket. The outer sheath is then removed. FIGS. 16A-D show an analogous arrangement for an IA which is supported/guided by three guidewires, for better stability. Numerous variations with different numbers, placements and sizes of guidewires are possible.

FIG. 17 shows a variation of the above-mentioned with a guidewire 1600 which loops in pocket 1620, leaving two protruding ends. IA 1602 accommodates both guidewire segments, and introduces sheath 1604 into the pocket.

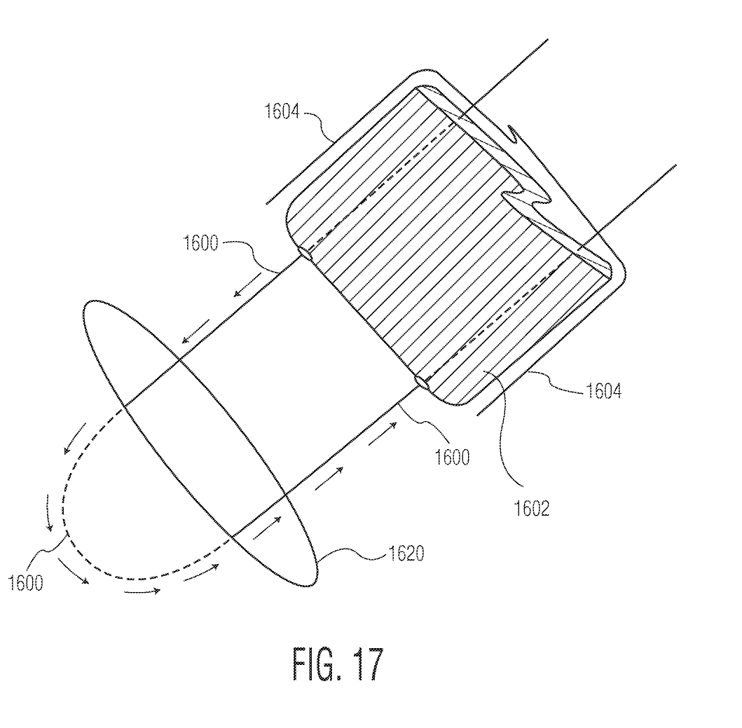

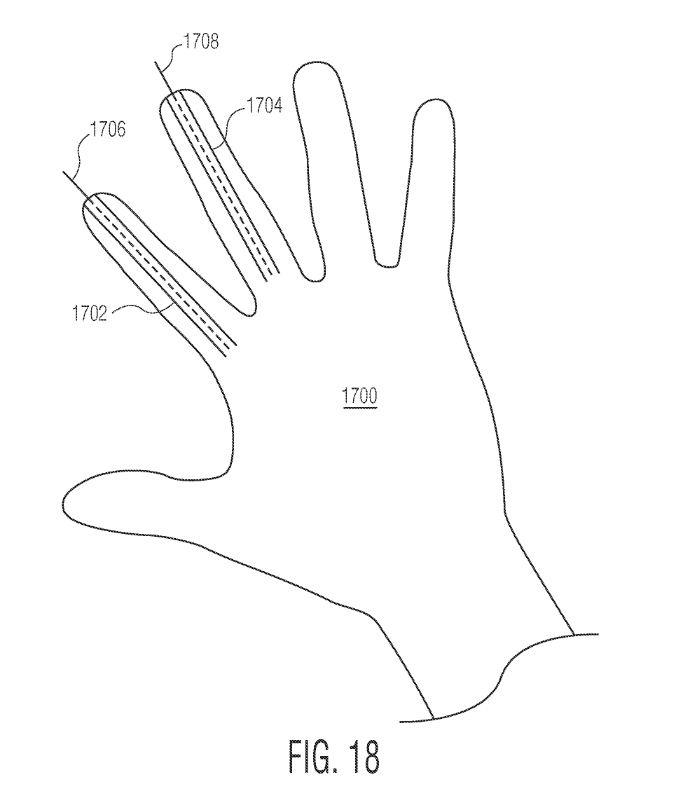

The tactile abilities and sensitivities of the fingers of a surgeon are a highly effective means of delivering and positioning a guidewire. FIG. 18 shows a glove with guiding receptacles 1702 and 1704 for guidewires 1706 and 1708. Numerous variations on this theme are possible, including guiding receptacles which are on the outside of the glove (“outboard”), the inside of the glove (“inboard”), or within the glove material itself. Numerous possible placements at the top of a glove finger, the bottom, sides, or in between fingers are possible. There may be one wire to a finger or glove, or many wires to a finger or glove.

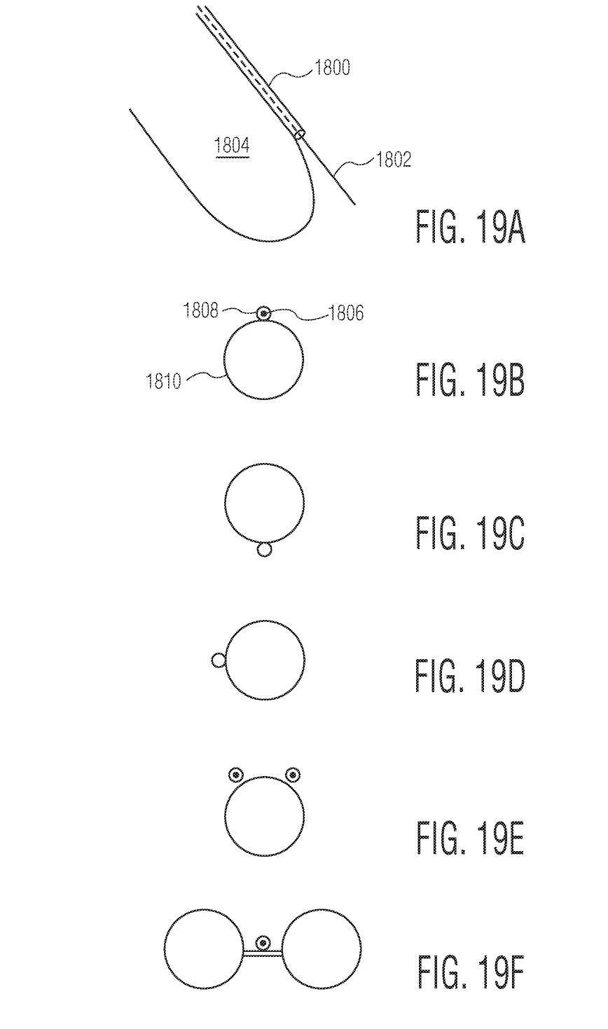

FIG. 19A shows glove finger 1804 with outboard guiding receptacle 1800 and guidewire 1802. FIG. 19B shows the guidewire 1806 in the receptacle 1808 at the top of the finger.

FIGS. 19C and 19D show positioning (without wire) at the bottom and laterally. FIG. 19E shows positioning, with one wire in each receptacle, for receptacles on one glove finger. FIG. 19F shows positioning between fingers.

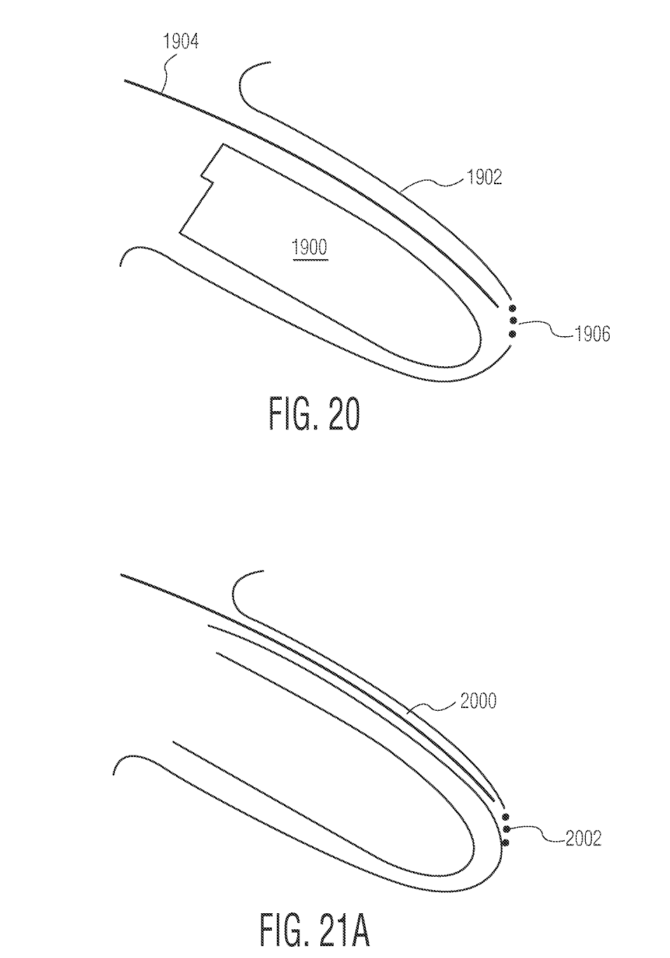

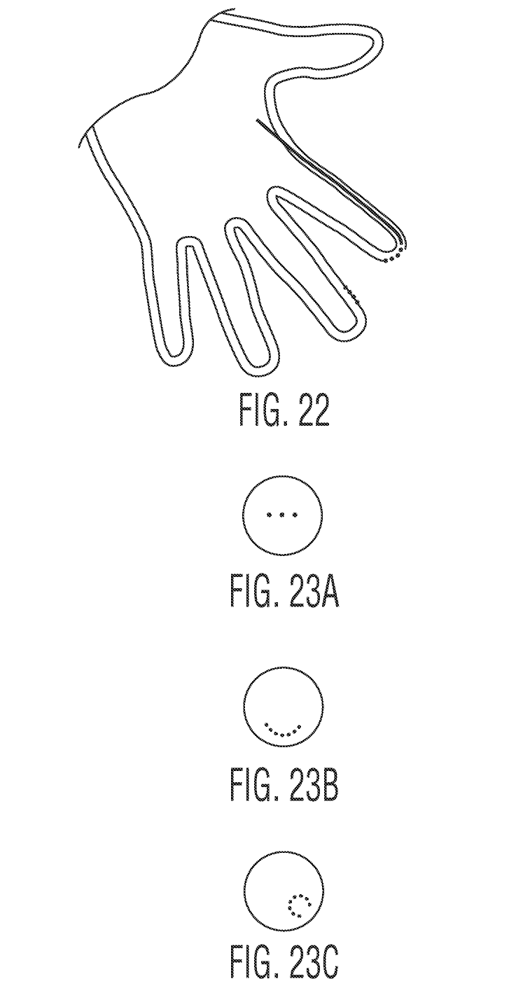

FIG. 20 shows an arrangement with an inner glove 1900 worn by the surgeon inside of outer glove or finger device 1902. This device does not have a separate guiding receptacle guidewire 1904 is advanced to tearable segment 1906, and the wire can be poked through to the outside to the outer glove. FIG. 22 shows various locations for the tearable segment, and FIGS. 23A-C shows various geometries of the tearable segment.

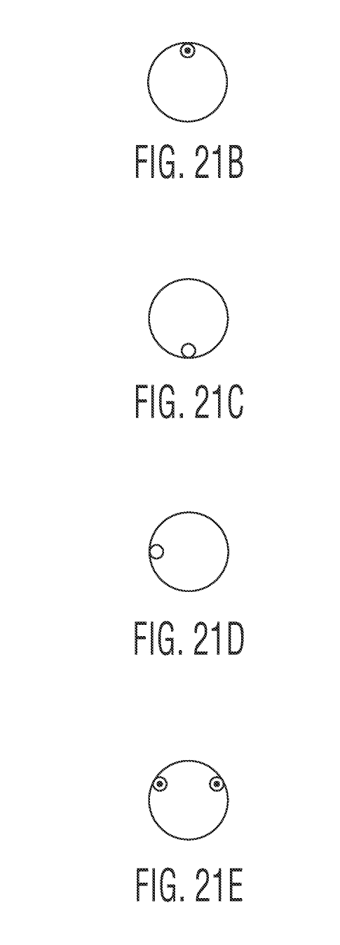

FIG. 21A shows a glove arrangement with an inboard guiding receptacle 2000 and a tearable segment 2002 at its distal end. FIGS. 21B to 21E show various positions for the inboard receptacles and are analogous to FIGS. 19B to 19E.

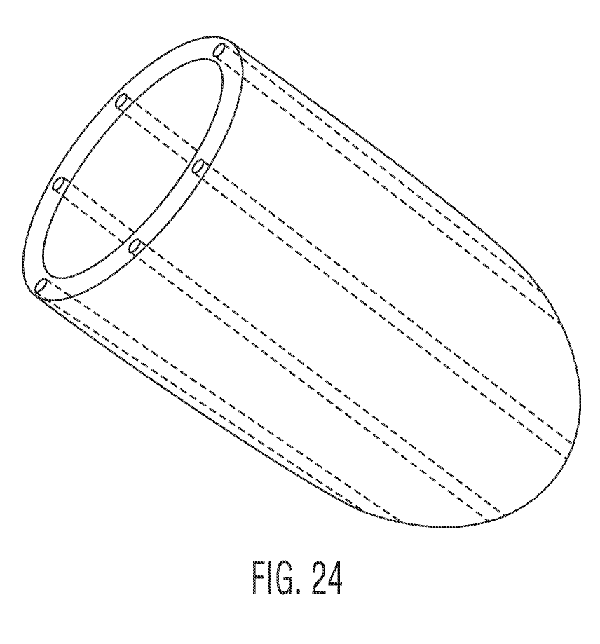

FIG. 24 shows a glove finger in which multiple guiding receptacles are within the glove material.

In each of the glove arrangements, attention to the sterile technique is executed, so that a surgeon's finger is never in contact with a guidewire or with any material that enters or traverses the body of a patient.

As mentioned hereinabove, numerous combinations of the aforementioned approaches, apparatus and methods will allow the surgeon to more efficiently insert and replace implantable medical devices in a living being.

Still other variations of the invention will be apparent to those skilled in the art, and are intended to be included in the disclosure of the medical device insertion and removal tool.

There has thus been shown and described a novel implantable medical device removal/insertion tool which fulfills all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

1. A medical device for guiding an implantable medical apparatus during its insertion into a cavity in a human body and for ensuring that the implantable medical apparatus is properly situated within the body, said medical device comprising:
(A) a first part adapted, for insertion into said cavity in the body, said first part having at least one surface which is configured to facilitate inserting and positioning said implantable apparatus in said cavity and removing the implantable apparatus from said cavity; and
(B) a second part having a distal end rigidly attached to the first part and having a proximal handle portion configured for gripping during use to insert and position the first part within said cavity and thereafter to remove the first part from said cavity;
whereby during use:
(1) said first part of the medical device is inserted into the cavity through an incision in said body;
(2) said first part guides the positioning of the implantable apparatus during at least one of
(i) the insertion of said implantable apparatus into said cavity;
(ii) the repositioning of said implantable apparatus within said cavity; and
(iii) the removal of said implantable apparatus from said cavity.

2. The medical device of claim 1, further comprising the implantable apparatus, wherein said implantable apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) a bladder stimulating device;
(l) an implantable receptor stimulating device;
(m) an implantable gastric stimulating device; and
(n) an implantable intestinal stimulating device.

3. The medical device of claim 1, wherein the proximal portion of said handle section is configured to be held by a gripper selected from the group consisting of (1) a hand of a surgeon; (2) a robotic arm of a robotic surgical apparatus; and (3) a surgical clamp.

4. The medical device of claim 1, wherein said first part comprises a substantially flat element configured to contact the implantable apparatus.

5. The medical device of claim 1, wherein said first part comprises a curved element configured to contact the implantable apparatus.

6. The medical device of claim 1, wherein at least one external contour of said first part is substantially similar to that of the implantable apparatus.

7. The medical device of claim 1, wherein said first part is substantially similar in its width to that of the implantable apparatus.

8. The medical device of claim 1, further comprising a hollow tube-shaped element which extends from a distal end of said first part to a proximal end of said first part, and a guiding device disposed in said tube-shaped element for facilitating the introduction of said implantable apparatus into said cavity;
whereby, in use:
(1) a distal portion of said guiding device is introduced into said cavity via said tube-shaped element; and
(2) said medical device is thereafter removed from said body by sliding it over said guiding device.

9. The medical device of claim 1, further comprising a hollow tube-shaped element which extends from a distal end of said first part to said handle portion, and a guiding device, disposed in said tube-shaped element, for facilitating the introduction of said implantable apparatus into said cavity;
whereby, in use:
(1) a distal portion of said guiding device is introduced into said cavity via said tube-shaped element; and
(2) said medical device is thereafter removed from said body by sliding it over said guiding device.

10. The medical device of claim 1, wherein said first part includes a clip device for removable attachment to the implantable apparatus.

11. The medical device of claim 2, wherein said implantable apparatus includes a clip device for removable attachment to the first part.

12. The medical device of claim 1, wherein the first part comprises first and second elements arranged substantially in parallel.

13. The medical device of claim 12, wherein said first and said second elements are spaced apart by a distance substantially equal to the thickness of the implantable apparatus.

14. The medical device of claim 12, wherein said first and said second elements are spaced apart by a variable distance which is in the range of from less than to greater than the width of the implantable apparatus.

15. The medical device of claim 14, wherein the distance between said first and said second elements is adjustable.

16. The medical device of claim 12, further comprising an attachment device situated at a proximal portion of each of said first and said second elements, for attaching said first element to said second element.

17. The medical device of claim 16, wherein said attachment device allows the detachment of the second element from said first part, wherein said detachment facilitates the removal of the implantable apparatus from said cavity, without the removal of at least the first element from said cavity.

18. The medical device of claim 16, wherein the second part comprises first and second elements substantially in parallel, configured such that
a distal end of the first element of the second part is rigidly attached to the first element of the first part, and
a distal end of the second element of the second part is rigidly attached to the second element of the first part
and wherein said attachment device allows for the rotation of each of said first element and said second element in parallel planes, each said plane being perpendicular to an axis of said attachment device;
whereby an increase in an angle determined by the orientation of each of the first and second elements of said second part causes an increase in an angle determined by the orientation of each of the first and second elements of said first part.

19. The medical device of claim 12, wherein at least one of the first and second elements of said first part is substantially flat.

20. The medical device of claim 12, wherein at least one of the first and second elements of said first part is curved, configured to contact the implantable apparatus.

21. The medical device of claim 12, wherein at least one external contour of at least one of said first and second elements of said first part is substantially similar to that of the implantable apparatus.

22. The medical device of claim 12, wherein at least one of said first and second elements of said first part is removably attachable to the implantable medical apparatus.

23. The medical device of claim 12, wherein the first part further comprises a third element disposed adjacent and arranged substantially parallel to the first and second elements, said third element being removably attachable to said first and second elements, thereby to allow removal of the first and second elements together with the implantable medical apparatus while the third element remains in said cavity to identify its location and retain the space.

24. The medical device of claim 12, wherein the first part further comprises a third element disposed adjacent and arranged substantially parallel to the first and second elements, said second and third elements being removably attachable to said first element, thereby to allow removal of the first element together with the implantable medical apparatus while the second and third elements remain in said cavity to identify its location and retain the space.

25. The medical device of claim 23, further comprising an attachment device situated at a proximal portion of each of said first, second and third elements, for attaching said third element to at least one of said first and said second elements, wherein said attachment device allows the detachment of the third element from said first part, and wherein said detachment facilitates the removal of the implantable apparatus from said cavity, without the removal of at least the third element from said cavity.

26. The medical device of claim 23, wherein at least one of the first, second and third elements of said first part is substantially flat.

27. The medical device of claim 23, wherein at least one of the first, second and third elements of said first part is curved and configured to contact the implantable apparatus.

28. The medical device of claim 23, wherein at least one external contour of at least one of said first, second and third elements of said first part is substantially similar to that of the implantable apparatus.

29. The medical device of claim 23, wherein at least one of said first, second and third elements of said first part are removably attachable to the implantable medical apparatus.

30. The medical device of claim 12, wherein the first part further comprises a third and a fourth element disposed adjacent and arranged substantially in parallel with the first and second elements, said third and fourth elements being removably attachable to said first and second elements, thereby to allow removal of the first and second elements together with the implantable medical apparatus while the third and fourth elements remain in said cavity to identify its location and retain the space.

31. The medical device of claim 30, wherein the third and fourth elements are arranged on opposite sides of said first and second elements.

32. The medical device of claim 30, wherein the third and fourth elements are arranged on one side of the first and second elements.

33. The medical device of claim 30, further comprising an attachment device situated at a proximal portion of each of said first, second, third and fourth elements, for attaching at least one of said third and said fourth element to at least one of said first and said second elements, wherein said attachment device allows the detachment of the third and fourth elements from said first part, and wherein said detachment facilitates the removal of the implantable apparatus from said cavity, without the removal of the third and fourth elements from said cavity.

34. The medical device of claim 30, wherein at least one of the first, second, third and fourth elements of said first part is substantially flat.

35. The medical device of claim 30, wherein at least one of the first, second, third and fourth elements of said first part is carved and configured to contact the implantable apparatus.

36. The medical device of claim 30, wherein at least one external contour of at least one of said first, second, third and fourth elements of said first part is substantially similar to that of the implantable apparatus.

37. The medical device of claim 30, wherein at least one of said first and said second elements of said first part are removably attachable to the implantable medical apparatus.

38. An article adapted to foe worn on the outside of at least one gloved finger of a surgeon implanting a medical apparatus in a cavity within a human body so as to facilitate inserting and positioning an inner guiding device in said cavity prior to implanting the medical apparatus,
said article comprising a first tube-shaped element, open at a proximal end and closed at a distal end, and being made of a thin deferrable material which allows the surgeon to sense the tissue contours on the inside of said cavity;
said article having a tearable line segment on said one end which is configured such that tearing of the material along the line segment does not result in a detachment of any material from said article;
whereby, during use, the inner guiding device can be introduced into said cavity of the body through a gap in the deformable material forming said tube-shaped element that is created by tearing said tearable line segment, thereby allowing the initial placement of the inner guiding device by the surgeon wearing said article.

39. The article of claim 38, wherein an internal surface of the tube-shaped element is slippery to allow the article to be placed on, and removed from, an outer surface of said at least one gloved finger and to facilitate the passage of the inner guiding device between said enter surface and the internal surface.

40. The article of claim 38, further comprising at least one additional inner tube-shaped element which extends from an open end of said first tube-shaped element to said tearable segment thereof, for accommodating the inner guiding device, wherein the inner guiding device is advanced to said tearable segment through said additional inner tube-shaped element.

41. The article of claim 40, wherein the internal surface of said additional inner tube-shaped element is slippery to facilitate the passage of the inner guiding device.

42. The article of claim 38, wherein the line segment is straight.

43. The article of claim 38, wherein the line segment is curved.

44. The article of claim 38, wherein the article comprises an outer glove having a hand portion adapted to be worn over a gloved hand of the surgeon and which is detachable from said tube-shaped element.

45. The article of claim 38, which comprises a plurality of said tube-shaped elements, each accommodating a respective gloved finger of said surgeon, each tube-shaped element being attached to at least one other tube-shaped element, and each of which has said tearable section;
wherein a plurality of inner guiding devices can be introduced into said cavity of the body, each through a gap in the deformable material forming said respective tube-shaped element that is created by tearing said tearable line segment.

46. The article of claim 40, which comprises a plurality of said additional inner tube-shaped elements, each of which extends from an open end of said first tube-shaped element to said tearable segment thereof, each for accommodating one of a plurality of said inner guiding device, wherein each said inner guiding device is advanced to said respective tearable segment through, said respective inner tuba-shaped element.

47. The article of claim 45, which comprises a plurality of said additional inner tube-shaped elements, each of which extends from an open end of one of said first tube-shaped elements to a respective tearable segment thereof, wherein each said inner guiding device is advanced to said respective tearable segment through said respective inner tube-shaped element.

48. The article of claim 38, further comprising the medical apparatus, wherein said medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) an implantable receptor stimulating device;
(m) an implantable gastric stimulating device; and
(n) an implantable intestinal stimulating device.

49. A system comprising:
(1) said article of claim 38; and
(2) said inner guiding device;
whereby, during use, a distal portion of said inner guiding device is positioned inside of said cavity and a proximal portion thereof is positioned outside of said cavity.

50. The system of claim 49, wherein said, inner guiding device is a guide wire.

51. The system of claim 50, wherein said guide wire is composed of a flexible material.

52. The system of claim 49, further comprising an intermediate guiding device with a hollow inner portion which accommodates said inner guiding device;
whereby, during use:
(1) said distal portion of said inner guiding device is introduced into said cavity,
(2) said article is thereafter removed from said body over said inner guiding device; and
(3) said intermediate guiding device is thereafter introduced into said cavity by sliding it over said inner guiding device.

53. The system of claim 49, further comprising at least one additional inner guiding device, and an intermediate guiding device with a plurality of hollow inner portions, each of which accommodates a respective inner guiding device;
whereby, during use:
(1) the distal portion of each of the plurality of said inner guiding devices is introduced into said cavity,
(2) said article is thereafter removed from said body over at least one of said inner guiding devices; and
(3) said intermediate guiding device is thereafter introduced into said cavity by sliding it over each of said plurality of inner guiding devices, each inner guiding device traversing one of said hollow inner portions.

54. The systems defined in claim 52, wherein the cross sectional dimensions of said intermediate guiding device are substantially similar to that of said medical apparatus, along at least one orientation of said medical apparatus.

55. The system defined in claim 52, wherein the cross sectional shape of said intermediate guiding device is substantially similar to that of said implanted apparatus, in at least, one orientation of said implanted apparatus.

56. The system defined in claim 52, further comprising an outer hollow guiding device, open at both ends, wherein said outer guiding device is adapted to accommodate in its hollow space said intermediate guiding device,
whereby, during use:
(1) a distal portion of said outer guiding device is introduced into said cavity over said intermediate guiding device;
(2) said intermediate guiding device is thereafter removed;
(3) said implantable apparatus is thereafter advanced into said cavity through the hollow portion of said outer guiding device; and
(4) said outer guiding device is thereafter removed from said cavity.

57. The system defined in claim 56, wherein the cross sectional dimensions of the hollow portion of said outer guiding device are substantially similar to that of said medical apparatus, along at least one orientation of said medical apparatus.

58. The system defined in claim 56, wherein the cross sectional shape of the hollow portion of said outer guiding device is substantially similar to that of said implanted apparatus, in at least one orientation of said implanted apparatus.

59. The system defined in claim 56, wherein the outer guide has a slippery coating on an inner surface thereof.

60. An article adapted to foe worn on the outside of at least one finger of a surgeon implanting a medical apparatus in a cavity within a human body so as to facilitate inserting and positioning an inner guiding device with a proximal end and a distal end in said cavity prior to implanting the medical apparatus,
said article comprising a first tube-shaped element for accommodating said finger, open at a proximal end and closed at a distal end, and being made of a thin deformable material which allows the surgeon to sense the tissue contours on the inside of said cavity;
said article having a second tube-shaped element for accommodating said inner guiding device, attached to said first tube-shaped element, and open at both a proximal end and a distal end, with a long axis approximately parallel to a long axis of said first tube-shaped element;
whereby, during use:
the introduction of said distal end of said first tube-shaped element into said cavity by said, surgeon introduces said distal end of said second tube-shaped element into said cavity;
said distal end of said inner guiding device is introduced into said proximal end of said second tube-shaped element and thereafter advanced sequentially to said distal end of said second tube-shaped portion and thence into said cavity; and
after removal of said article from said cavity without the removal of said distal end of said inner guiding device from said cavity, said inner guiding device is positioned with said distal end inside of said cavity and said proximal end outside of said, cavity;
thereby facilitating surgical access to said cavity.

61. The article of claim 60, wherein said article is a surgical glove having a plurality of first tube-shaped elements, each for accommodating one finger, and wherein said second tube-shaped element is attached to one of said plurality said tube-shaped elements.

62. The article of claim 61, wherein said surgical glove comprises a plurality of second tube-shaped elements, each second tube-shaped element accommodating a respective inner guiding device, each second tube-shaped element attached to at least one of said first tube-shaped elements, each second tube-shaped element open at both a proximal end and a distal end, with a long axis approximately parallel to a long axis of said first tube-shaped element;
whereby, during use:
the introduction of said distal end of said at least one first tube-shaped element into said cavity by said surgeon introduces said plurality of distal ends of said second tube-shaped elements into said cavity;
said distal ends of said inner guiding device are introduced into said respective proximal ends of said second tube-shaped elements and thereafter advanced sequentially to said respective distal ends of said second tube-shaped elements and thence into said cavity;
after removal of said article from said cavity without the removal of said distal ends of said inner guiding devices from said cavity, said inner guiding devices are positioned with said distal ends inside of said cavity and said proximal ends outside of said cavity;
thereby facilitating surgical access to said cavity.

63. The article of claim 60, wherein the internal surface of said second tube-shaped element is slippery to facilitate the passage of the inner guiding device.

64. The article of claim 60, further comprising the medical apparatus, wherein said medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) as implantable muscle stimulating device;
(k) an implantable bladder stimulating device;
(l) an implantable receptor stimulating device;
(m) an implantable gastric stimulating device; and
(n) an implantable intestinal stimulating device.

65. A system comprising
(1) said article of claim 60; and
(2) said, inner guiding device;
whereby, during use, a distal portion of said inner guiding device is positioned inside of said cavity and a proximal portion thereof is positioned outside of said cavity.

66. The system of claim 65, wherein said inner guiding device is a guide wire.

67. The system of claim 66, wherein said inner guide wire is composed of a flexible material.

68. The system of claim 66, further comprising an intermediate guiding device with a hollow inner portion which accommodates and surrounds said inner guiding device;
whereby, during use:
(1) said distal portion of said inner guiding device is introduced into said cavity,
(2) said article is thereafter removed from said body and from said inner guiding device by sliding said article over said inner guiding device; and
(3) said intermediate guiding device is thereafter introduced into said cavity by sliding it over said inner guiding device.

69. The system of claim 66, wherein said intermediate guiding device further comprises a plurality of hollow inner portions, each of which accommodates an inner guiding device;
whereby, during use:
(1) the distal portion of each of a plurality of said inner guiding devices is introduced into said cavity,
(2) said article is thereafter removed from said body and from each of said plurality of inner guiding devices by sliding said article over each of said plurality of inner guiding devices; and
(3) said intermediate guiding device is thereafter introduced into said cavity by sliding it over each of said plurality of inner guiding devices, each inner guiding device traversing one of said hollow inner portions.

70. The system defined in claim 68, wherein the cross sectional dimensions of said intermediate guiding device are substantially similar to that of said medical apparatus, along at least one orientation of said medical apparatus.

71. The system defined in claim 68, wherein the cross sectional shape of said intermediate guiding device is substantially similar to that of said medical apparatus, in at least one orientation of said medical apparatus.

72. The system defined in claim 68 further comprising an outer hollow guiding device, open at both ends, wherein said outer guiding device is adapted to accommodate in its hollow space said intermediate guiding device, wherein
(1) a distal portion of said outer guiding device is introduced into said cavity over said intermediate guiding device;
(2) said intermediate guiding device is thereafter removed;
(3) said medical apparatus is thereafter advanced into said cavity through the hollow portion of said outer guiding device; and
(4) said outer guiding apparatus is thereafter removed from said cavity.

73. The system defined in claim 72, wherein the cross sectional dimensions of the hollow portion of said outer guiding device are substantially similar to that of said medical apparatus, along at least one orientation of said medical apparatus.

74. The system defined in claim 72, wherein the cross sectional shape of the hollow portion of said outer guiding device is substantially similar to that of said medical apparatus, in at least one orientation of said medical apparatus.

75. A medical device for facilitating the insertion and positioning of an implantable medical apparatus in a cavity within a human body, said medical device comprising:
a) a first guiding device having a proximal end and a distal end configured for insertion into said cavity;
b) a first implanting device having a cross-sectional shape approximating that of said medical apparatus and comprising an inner hollow shaft with a width slightly larger than that of said first guiding device, for accommodating and surrounding said first guiding device, said implanting device having a distal end configured for insertion into said cavity and having a proximal end; and
c) a first hollow sheath having a cross-sectional shape approximating that of a cross-sectional shape of said medical apparatus and said first implanting device, and having an inner opening with a width at least as large as the width of said first implanting device;
whereby, during use:
(1) the distal end of said first guiding device is inserted into said cavity;
(2) said first sheath, with said first implanting device disposed in said inner opening, is passed over said first guiding device into said cavity; and
(3) said first guiding device and said first implanting device are thereafter removed from said sheath;
thereby facilitating the insertion of said implantable medical apparatus into said cavity.

76. The medical device of claim 75, wherein said first sheath is configured to tear away after insertion and positioning of said medical apparatus.

77. The medical device of claim 75, wherein said first sheath is at least as large as said medical apparatus, thereby facilitating the insertion of said medical apparatus into said cavity.

78. The medical device of claim 75, further comprising
a) a second guiding device having a larger width than that of said first guiding device, having a distal end configured for insertion into said cavity and having a proximal end;
b) a second implanting device having a larger width than that of said first implanting device, having a cross-sectional shape approximating that of said medical apparatus and comprising an inner hollow shaft with a width slightly larger than that of said second guiding device, for accommodating and surrounding said second guiding device, said second implanting device having a distal end configured for insertion into said cavity, and having a proximal end; and
c) a second hollow sheath having a cross-sectional shape approximating that of a cross-sectional shape of said medical apparatus and said second implanting device, and having an inner opening with a width at least as large as the width of said second implanting device;
whereby, during use:
(1) the distal end of said second guiding device is inserted into said first sheath;
(2) said first sheath is thereafter removed;
(3) said second sheath, with said second implanting device disposed in said inner opening, is thereafter passed over said second guiding device into said cavity;
(4) said second guiding device and said second implanting device are thereafter removed from said second sheath;
(5) said implantable medical apparatus is thereafter inserted through said second sheath into said cavity; and
(6) said second sheath is thereafter removed from said cavity;
thereby facilitating the insertion of said implantable medical apparatus into said cavity.

79. The medical device of claim 75, further comprising at least one additional guiding device and wherein said implanting device contains one additional hole for each respective additional guidewire.

80. The medical device of claim 75, further comprising the medical apparatus, wherein said medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) an implantable receptor stimulating device;
(m) an implantable gastric stimulating device; and
(n) an implantable intestinal stimulating device.

81. A balloon device for insertion into a cavity of a human body to facilitate the placement of an implantable medical apparatus in the cavity, said balloon device comprising:
a balloon having a closable mouth which surrounds a valve apparatus for controlling the ingress and egress of a fluid;
a tube-shaped element open at both ends and having a side attached to an outer surface of said balloon, to accommodate and surround a guiding device, said guiding device having a distal end and a proximal end;
whereby, during use:
(1) the distal mid of the guiding device is inserted into the cavity;
(2) thereafter, the tube-shaped element is passed over said guiding device and inserted with said balloon into said cavity;
(3) thereafter, said guiding device is removed from said tube-shaped element;
(4) thereafter, said balloon is inflated via said valve;
(5) thereafter, said implantable medical device is inserted into said cavity and said balloon is deflated via said valve; and
(6) thereafter, said deflated balloon is removed from said cavity.

82. The balloon device of claim 81, wherein an exterior surface of said balloon is coated with a slippery substance, thereby to facilitate the removal of said balloon from said cavity following deflation.

83. The balloon device of claim 81, wherein, following the inflation of the balloon, at least one of (i) the shape, and (ii) the size, of said balloon approximates that of an implantable medical device.

84. The balloon device of claim 83, wherein at least one of said shape and said size is selected from the group consisting of:
(a) the shape and size of an implantable pacemaker;
(b) the shape and size of an implantable defibrillator;
(c) the shape and size of an implantable loop recorder;
(d) the shape and size of an implantable cardiac resynchronization device;
(e) the shape and size of an implantable nerve stimulating device;
(f) the shape and size of an implantable spinal cord stimulating device;
(g) the shape and size of an implantable brain stimulating device;
(h) the shape and size of an implantable medication delivery device;
(i) the shape and size of an implantable glucose monitoring device;
(j) the shape and size of an implantable muscle stimulating device;
(k) the shape and size of an implantable bladder stimulating device;
(l) the shape and size of en implantable receptor stimulating device;
(m) the shape and size of an implantable gastric stimulating device; and
(n) the shape and size of an implantable intestinal stimulating device.

85. The balloon device of claim 81, further comprising the implantable medical apparatus, wherein said medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) an implantable receptor stimulating device;
(m) an implantable gastric stimulating device; and
(n) an implantable intestinal stimulating device.

86. The balloon device of claim 81, further comprising at least one projection, attached to said outer surface of said balloon, for facilitating the removal of said balloon from said cavity, wherein at least a portion of said projection remains outside of said cavity when said balloon is inside of said cavity.

87. The balloon device of claim 81, further comprising the guiding device, and wherein said guiding device is a guide wire.

88. A balloon device for insertion into a cavity of a human body to facilitate the placement of an implantable medical apparatus in the cavity, said balloon device comprising:
a balloon having a proximal end with a closable opening through which an implantable medical device may be inserted, and having a distal end;
a valve apparatus, coupled to said balloon, for controlling the ingress and egress of a fluid into and out of said balloon;
a tube-shaped element open at both ends and having a side attached to an outer surface of said balloon to accommodate and surround a guiding device, said guiding device having a distal end and a proximal end; and
a tearable segment on said balloon to facilitate removing said balloon from said body after said placement;
whereby, during use,
(1) the distal end of the guiding device is inserted into the cavity;
(2) thereafter, the distal end of said tube-shaped element is passed over the proximal end of said guiding device and is inserted with said balloon into said cavity;
(3) thereafter, said guiding device is removed from said tube-shaped element;
(4) said implantable medical apparatus is inserted info the closable opening of said balloon and thereafter said opening is closed;
(5) thereafter, said balloon is inflated via said valve;
(6) thereafter, said implantable medical device is advanced into the distal end of said balloon situated within said cavity; and
(7) thereafter said balloon is deflated, said tearable segment is torn and said balloon is removed from said cavity.

89. The balloon device of claim 88, wherein an exterior surface of said balloon is coated with a slippery substance, thereby to facilitate the removal of said balloon from said cavity following deflation.

90. The balloon device of claim 88, wherein, following the inflation of the balloon, at least one of (i) the shape, and (ii) the size, of said balloon approximates that of an implantable medical device.

91. The balloon device of claim 90, wherein at least one of said shape and said size is selected from the group consisting of:
(a) the shape and size of an implantable pacemaker;
(b) the shape and size of an implantable defibrillator;
(c) the shape and size of an implantable loop recorder;
(d) the shape and size of an implantable cardiac resynchronization device;
(e) the shape and size of an implantable nerve stimulating device;
(f) the shape and size of an implantable spinal cord stimulating device;
(g) the shape and size of an implantable brain stimulating device;
(h) the shape and size of an implantable medication delivery device;
(i) the shape and size of an implantable glucose monitoring device;
(j) the shape and size of an implantable muscle stimulating device;
(k) the shape and size of an implantable bladder stimulating device;
(l) the shape and size of an implantable receptor stimulating device;
(m) the shape and size of an implantable gastric stimulating device; and
(n) the shape and size of an implantable intestinal stimulating device.

92. The balloon device of claim 88, further comprising at least one projection, attached to said outer surface of said balloon, for facilitating the removal of said balloon from said cavity, wherein at least a portion of said projection remains outside of said cavity when said balloon is inside of said cavity.

93. The balloon device of claim 88, further comprising said guiding device, and wherein said guiding device is a guide wire.

94. The balloon device of claim 88, wherein at least part of said tearable segment is situated at the distal end of said balloon.

95. The balloon device of claim 88, wherein at least part of said tearable segment is situated at the proximal end of said balloon.

96. The balloon device of claim 88, further comprising the implantable medical apparatus, wherein said medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) an implantable receptor stimulating device;
(m) an implantable gastric stimulating device; and
(n) an implantable intestinal stimulating device.

97. An implantable medical device with a casing including at least one tubular region which is open at a distal end and extends to a proximal open end, said tubular region accommodating and surrounding a guiding device having a distal end and a proximal end, said guiding device facilitating the insertion of said medical device into a cavity in a human body;
whereby, during use:
(1) the distal end of said guiding device is inserted into said cavity;
(2) the proximal end of said guiding device is thereafter inserted into the distal end of said tubular region of said implantable device, and said implantable device is thereafter advanced over said guiding device into said cavity; and
(3) thereafter, said guiding device is removed from said cavity, leaving said implantable device in said cavity.

98. The implantable device of claim 97, wherein at least one of said tubular regions abuts an outer wall of said implantable device.

99. The implantable device of claim 97, wherein at least one of said tubular regions does not abut an outer wall of said implantable device.

100. The implantable device of claim 98, wherein said tubular region is detachable from said implantable device.

101. The implantable device of claim 97, wherein said implantable device is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) as implantable receptor stimulating device;
(m) an implantable gastric stimulating device; and
(n) an implantable intestinal stimulating device.

102. The implantable device of claim 97, further comprising said guiding device, and wherein said guiding device is a guide wire.

103. A stretchable belt adapted to be removably attached around a perimeter of an implantable medical apparatus for facilitating the implantation of the medical apparatus into a cavity of a human body, said belt comprising:
(a) two end segments connected together via an elastic intermediate segment;
(b) a fastening device for joining the two end segments with said belt encircling the medical apparatus, in such a manner as to reversibly maintain a state of tension in said belt; and
(c) at least one tubular guiding receptacle, said tubular receptacle accommodating and surrounding a guiding device, said guiding device having a distal end and a proximal end, said guiding device facilitating the insertion of said medical device into a cavity in a human body;
whereby, during use:
(1) the distal end of said guiding device is inserted into said cavity;
(2) the belt is positioned to encircle the implantable medical apparatus with said fastening device maintaining sufficient tension to cause close adherence of said belt to said implantable medical apparatus;
(3) the implantable medical apparatus is advanced over the proximal end of said guiding device into said cavity; and
(4) thereafter, said belt is detached from said implantable medical apparatus and said guiding device and said belt are removed from said cavity.

104. The belt of claim 103, wherein an axis of said tabular guiding receptacle is parallel to the plane defined by said belt when said belt encompasses said implantable medical apparatus;
whereby the belt has a longitudinal orientation with respect to a direction of insertion of the implantable medical apparatus.

105. The belt of claim 103, wherein an axis of said tabular guiding receptacle is perpendicular to the plane defined by said belt when said belt encompasses said implantable medical apparatus;
whereby the belt has a transverse orientation with respect to a direction of insertion of the implantable medical apparatus.

106. The belt of claim 103, further comprising the implantable medical apparatus, wherein, said implantable medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) an implantable receptor stimulating device;
(m) an implantable gastric stimulating device; and
(n) an implantable intestinal stimulating device.

107. A system comprising:
(1) said belt of claim 103; and
(2) said guiding device;
whereby, during use, when the belt encircling the implantable medical apparatus is inserted in said cavity, a distal portion of said guiding device is positioned inside of said cavity and a proximal portion thereof is positioned outside of said cavity.

108. A system fax inserting and positioning an implantable medical apparatus inside of a cavity in a body of a human, said system comprising:
(A) a flexible guide wire having a proximal end, a mid-section and a distal end;
(B) an intermediate guiding device having a proximal end, a distal end and two hollow inner portions, each hollow portion extending from said proximal end to said distal end, for accommodating and surrounding said flexible guide wire; and
(C) an outer hollow guiding device, open, at both ends, adapted to accommodate and surround said intermediate guiding device in a hollow space therein;
whereby, during use:
(1) the proximal end of said guide wire is inserted into said cavity;
(2) thereafter, said guide wire is further advanced into said cavity until both said proximal end and said distal end each protrude from said cavity, while at least a portion of said mid-section lies inside of said cavity;
(3) thereafter, the proximal end and the distal end of said guide wire are inserted into separate hollow inner portions at the distal end of said intermediate gliding device, and advanced to the respective proximal end of said hollow inner portions;
(4) said outer guiding device, with said intermediate guiding device disposed in said hollow space of said outer guiding device, is advanced into said cavity over said flexible; guide wire;
(5) said guide wire and said intermediate guiding device are thereafter removed from said cavity; and
(6) said implantable medical apparatus is placed into said cavity via said outer guiding device.

109. The system defined in claim 108, wherein at least one of (i) the cross sectional dimensions, and (ii) the cross sectional shape of said intermediate guiding device are substantially the same as that of said implantable medical apparatus, along at least one orientation of said implantable medical apparatus.

110. The system defined in claim 108, wherein at least one of (i) the cross sectional distensions, and (ii) the cross sectional shape of said outer hollow guiding device are substantially the same as that of said implantable medical apparatus, along at least one orientation of said medical device.

111. The system defined in claim 108, further comprising the implantable medical apparatus, wherein said implantable medical apparatus is selected from the group consisting of:
(a) an implantable pacemaker;
(b) an implantable defibrillator;
(c) an implantable loop recorder;
(d) an implantable cardiac resynchronization device;
(e) an implantable nerve stimulating device;
(f) an implantable spinal cord stimulating device;
(g) an implantable brain stimulating device;
(h) an implantable medication delivery device;
(i) an implantable glucose monitoring device;
(j) an implantable muscle stimulating device;
(k) an implantable bladder stimulating device;
(l) an implantable receptor stimulating device;
(m) an implantable gastric stimulating device; and
(n) an implantable intestinal stimulating device.
